(19) United States
(12) Patent Application Publication
(10) Pub. No.: US 2025/0154123 A1
(43) Pub. Date: May 15, 2025

(54) COOLING COMPOUNDS AND COMPOSITIONS

(71) Applicant: V. MANE FILS, Le Bar-Sur-Loup (FR)

(72) Inventor: Fabien GRASSET, Peymeinade (FR)

(21) Appl. No.: 18/726,907

(22) PCT Filed: Jan. 6, 2023
(86) PCT No.: PCT/EP2023/050213

§ 371 Date: Jul. 5, 2024

(30) Foreign Application Priority Data
Jan. 7, 2022 (EP) 22150584.5

(51) Int. Cl. C07D 319/08; A23G 4/06; C07C 31/27; C07C 43/115; C07C 47/32; C07C 69/16; C07C 69/40; C07D 405/04; C07D 409/04

(52) U.S. Cl. CPC C07D 319/08; A23G 4/06; C07C 31/276; C07C 43/115; C07C 47/32; C07C 69/16; C07C 69/40; C07D 405/04; C07D 409/04

(57) ABSTRACT

Embodiments of the invention are directed to cooling compounds and cooling compositions having a unique cooling perception, as well as various edible, potable, and/or cosmetic products containing one or more cooling compounds having a general Formula (I): wherein Q is selected from CR¹R² or CH-CH(CH₂OR⁸)CH₂OR⁹; wherein R¹ and R² are independently selected from the group consisting of CHO, CO₂H; CH₂CH₂OH; CHR¹⁰OR¹¹, and CHR¹⁰OR¹²; wherein R⁸ and R⁹ are independently H, or in combination form a spiroacetal or spiroketal moiety; wherein each R¹⁰ is independently H or CH₃; wherein R¹¹ and R¹² are independently selected from H, substituted or unsubstituted C1 to C6 alkyls, substituted or unsubstituted cycloalkyls, substituted or unsubstituted alkaryls, substituted or unsubstituted aryls or heteroaryls, and substituted or unsubstituted acyls, or wherein R¹¹ and R¹² in combination form a spiroketal or a spiroacetal moiety; wherein R³, R⁴, R⁵, and R⁶ are independently selected from H, CH₃, and CH₂CH₃; and wherein R⁷ is selected from H, iso-propyl, isopropenyl, and sec-butyl; with the proviso that R³, R⁴, R⁵, R⁶, and R⁷ are not all H.

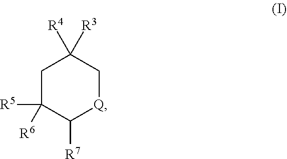

### FIELD OF THE INVENTION

This invention relates to cooling compounds and compositions having a unique cooling perception that provides the user with a pleasing cooling effect without bitterness. The invention also relates to products containing one or more cooling compounds.

### BACKGROUND OF THE INVENTION

A variety of chemical compounds are known which provide a cooling sensation when ingested or contacted with the body. Perhaps the best known of these compounds is menthol, which presumably acts on the cold receptors at the nerve endings in order to provide this cooling effect.

Since menthol has a strong minty odor and high relative volatility, new compounds have been developed and reported as potential flavorants or odorants useful in a variety of topical and ingestible compositions. For example, U.S. Pat. No. 5,009,893 proposes the use of menthol in combination with N-substituted-p-menthane carboxamide compounds as coolant compositions in edible products.

International Patent application publication No. WO93/23005 proposes coolant compositions for edible or topical products that comprise a ketal and a secondary coolant, which may be selected from menthol, carboxamides and mixtures thereof. In addition, this patent application mentions several other references which disclose compounds which have a flavor resembling menthol including menthyl carbinol, saccharide esters of menthol and a variety of amides. Also 2,3-p-menthane diol has been reported as having a sharp cooling taste.

German Patent application 2 339 661 discloses aromatic compositions which include menthol or menthol esters of heterocyclic carboxylic acids. The preferred ester is menthyl-2-pyrrolidone-5-carboxylic acid ester.

German Patent application 26 08 226 discloses a composition which exhibits a physiological cooling effect. The cooling compounds disclosed include menthol esters of naturally occurring hydroxycarboxylic acids having 2-6 carbon atoms, which are esterified with a C₁-C₄ alkyl group. Menthyl acetate and menthyl lactate are the most preferred cooling compounds of this disclosure.

International Patent application publication No. WO97/07771A1 discloses refreshing compositions, flavoring compositions and ingestible and topical compositions containing monomenthyl succinate, alkaline metal or alkaline-earth metal salts of monomenthyl succinate, and mixtures thereof. Secondary coolant agents which may be used in combination with the monomenthyl succinate and/or its salts include menthol, carboxamides, ketals, menthyl acetate, menthyl lactate, 3-menthoxypropane-1,2 diol, and mixtures thereof.

Finally, European patent EP80148B1 describes 3-menthoxypropane-1,2 diol, which is another commercially-available compound having cooling properties.

Thus, a variety of compounds are known to possess cooling properties and are useful in a wide variety of products. However, there is still a need to provide new cooling compositions having an improved cooling effect and/or taste perception.

Accordingly, it is an object of the present invention to provide improved cooling compositions. Another object of the present invention is to provide cooling compositions having a unique cooling sensation and taste perception. And yet another object of the present invention is to provide cooling compositions including two or more cooling agents, which can provide a complementary cooling sensation and taste perception.

These and other objects of the present invention will be apparent from the detailed description and examples that follow.

### SUMMARY OF THE INVENTION

Certain aspects of the present disclosure are described in the appended claims. There are additional features and advantages of the subject matter described herein. They will become apparent as this specification proceeds. In this regard, it is to be understood that the claims serve as a brief summary of varying aspects of the subject matter described herein. The various features described in the claims and below for various embodiments may be used in combination or separately. For example, specified ranges may be inclusive of their recited endpoints, unless explicitly excluded. Any particular embodiment need not provide all features noted above, nor solve all problems or address all issues noted above.

In accordance with embodiments of the present invention, a cooling composition is provided comprising (or consisting essentially of) an effective amount of a cooling compound having a general Formula (I):

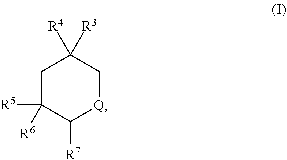

wherein Q is selected from CR¹R² or CH-CH(CH₂OR⁸)CH₂OR⁹; wherein R¹ and R² are independently selected from the group consisting of CHO, CO₂H; CH₂CH₂OH; CHR¹⁰R¹¹, and CHR¹⁰OR¹²; wherein R⁸ and R⁹ are independently H, or in combination form a spiroacetal or spiroketal moiety; wherein each R¹⁰ is independently H or CH₃; wherein R¹¹ and R¹² are independently selected from H, substituted or unsubstituted C1 to C6 alkyls, substituted or unsubstituted cycloalkyls, substituted or unsubstituted alkaryls, substituted or unsubstituted aryls or heteroaryls, and substituted or unsubstituted acyls, or wherein R¹¹ and R¹² in combination form a spiroketal or a spiroacetal moiety;
wherein R³, R⁴, R⁵, and R⁶ are independently selected from H, CH₃, and CH₂CH₃; and
wherein R⁷ is selected from H, iso-propyl, isopropenyl, and sec-butyl; with the proviso that R³, R⁴, R⁵, R⁶, and R⁷ are not all H; and
an edible, potable, or cosmetic ingredient.

Embodiments of the present invention further relate to a product composition selected from topical, ingestible, or tobacco products, such as oral care products, nasal care products, toiletries, filters, combustible papers and coating sheets for smoking tobacco products, chewing tobacco, chewing tobacco products, snuff tobacco products, chewing gum and chewing gum products, comprising:
a topical, ingestible, or tobacco base product, and
an effective amount of a cooling composition comprising (or consisting essentially of) the cooling compound having the general Formula (I).

Embodiments of the present invention also further relate to use of the cooling compounds having the general Formula (I) as a cooling agent or a perfume agent.

In accordance with yet another embodiment of the present invention, a cooling compound is provided having a general Formula (I):

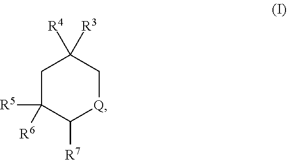

wherein Q is selected from CR¹R² or CH-CH(CH₂OR⁸)CH₂OR⁹; wherein R¹ is selected from the group consisting of CHO, CO₂H; CH₂CH₂OH; CHR¹⁰R¹¹; wherein R² is selected from the group consisting of CHO, CH₂CH₂OH, and CHR¹⁰OR¹²; wherein R⁸ and R⁹ are independently H, or in combination form a spiroacetal or spiroketal moiety; wherein R¹⁰ is H or CH₃; wherein R¹¹ and R¹² are independently selected from H, substituted or unsubstituted C1 to C6 alkyls, substituted or unsubstituted cycloalkyls, substituted or unsubstituted alkaryls, substituted or unsubstituted aryls or heteroaryls, and substituted or unsubstituted acyls, or wherein R¹¹ and R¹² in combination form a spiroketal or a spiroacetal moiety; and
wherein R³, R⁴, R⁵, and R⁶ are independently selected from H, CH₃, and CH₂CH₃; where R⁷ is selected from iso-propyl, isopropenyl, and sec-butyl;
   with a first proviso that R³, R⁴, R⁵, R⁶, and R⁷ are not all H;
   with a second proviso that when Q is CR¹R², and R¹ and R² are both CH₂OH:
      then R⁷ is not H; and
      then R³, R⁵, and R⁶ are not all H, R⁴ is not CH₃, and R⁷ is not isopropyl;
   with a third proviso that when Q is CR¹R², R¹ and R² are both CH₂OH, and R⁷ is isopropyl or isobutyl, then at least two or more of R³, R⁴, R⁵, and R⁶ are CH₃ or CH₂CH₃; and with a fourth proviso that when Q is CR¹R², and R¹ is CHR¹⁰R¹¹, R² is CHR¹⁰OR¹², R¹⁰ is H, and R¹¹ and R¹² in combination form a spiroketal or a spiroacetal moiety, and R⁷ is H, then at least two or more of R³, R⁴, R⁵, and R⁶ are CH₃ or CH₂CH₃.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1 is a graph showing preliminary comparative cooling intensity of several dilute aqueous solutions of Compound 1 (((2S,5R)-2-isopropyl-5-methylcyclohexane-1,1-diyl)dimethanol) and Compound 2 ((7S,10R)-7-isopropyl-3,10-dimethyl-2,4-dioxaspiro[5.5]undecane) against WS-3™ and WS-5™ over time.

FIG. 2 is a graph showing comparative evaluation of Compound 1 (((2S,5R)-2-isopropyl-5-methylcyclohexane-1,1-diyl)dimethanol) against menthol, WS-3™, PHYSCOOL™, and mineral water over time.

FIG. 3 is a graph showing comparative evaluation of Compound 1 (((2S,5R)-2-isopropyl-5-methylcyclohexane-1,1-diyl)dimethanol) versus PHYSCOOL™ over 10 minutes.

### DETAILED DESCRIPTION

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” “at least one,” and “the” include plural referents unless context clearly indicates otherwise. Similarly, the word “or” is intended to include “and” unless the context clearly indicates otherwise. The term “comprising” means “including;” hence, “comprising A or B” means including A or B, as well as A and B together.

In accordance with embodiments of the present invention, a cooling composition is provided comprising (or consisting essentially of) an effective amount of a cooling compound having a general Formula (I):

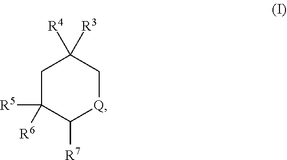

wherein Q is selected from CR¹R² or CH-CH(CH₂OR⁸)CH₂OR⁹; wherein R¹ and R² are independently selected from the group consisting of CHO, CO₂H; CH₂CH₂OH; CHR¹⁰OR¹¹, and CHR¹⁰OR¹²; wherein R⁸ and R⁹ are independently H, or in combination form a spiroacetal or spiroketal moiety; wherein each R¹⁰ is independently H or CH₃; wherein R¹¹ and R¹² are independently selected from H, substituted or unsubstituted C1 to C6 alkyls, substituted or unsubstituted cycloalkyls, substituted or unsubstituted alkaryls, substituted or unsubstituted aryls or heteroaryls, and substituted or unsubstituted acyls, or wherein R¹¹ and R¹² in combination form a spiroketal or a spiroacetal moiety;
wherein R³, R⁴, R⁵, and R⁶ are independently selected from H, CH₃, and CH₂CH₃; and
wherein R⁷ is selected from H, iso-propyl, isopropenyl, and sec-butyl; with the proviso that R³, R⁴, R⁵, R⁶, and R⁷ are not all H; and
an edible, potable, or cosmetic ingredient.

As used herein, “an effective amount” means a quantity of the cooling compound or cooling composition that imparts a statistically noticeable cooling effect to the composition or the product, as determined by a person having ordinary skill in the flavor arts.

As used herein, “cooling compound” means that the compound is active toward hTRPM8 receptors and provides a cooling effect on skin and/or mucosa. In some instances, the cooling compound may also possess olfactory chemoreceptor activity, thereby also making said compounds useful as fragrance molecules. Accordingly, depending on the inherent characteristics of each cooling compound disclosed herein, the cooling compound may be used as a cooling agent and/or a perfume agent.

As used herein, “substituted” means containing a functional group selected from an ester, an acid, or an alkoxy moiety.

C1 to C6 alkyl means a linear or branched alkyl group comprising from 1 to 6 carbon atoms, such as methyl, ethyl, n-propyl, isopropyl, n-butyl, iso-butyl, sec-butyl, pentyl, and hexyl.

Cycloalkyl means a monocyclic, saturated hydrocarbon group of the formula CₙH₂ₙ₋₁, such as (but not limited to) cyclopropyl, cyclobutyl, cyclopentyl, cyclohexyl, and cycloheptyl. Unless otherwise specified, a cycloalkyl group comprises from 3 to 8 carbon atoms.

Aryl means an aromatic ring comprising from 5 to 10 carbon atoms, consisting of one ring or several fused rings. Aryl is preferably a phenyl.

Heteroaryl means a heteroaromatic ring comprising from 4 to 10 carbon atoms, and from 1 to 3 heteroatoms chosen from O, S or N. Non-limiting examples include pyridinyl, thiophenyl, or furanyl.

Alkaryl means the radical Alk-Ar wherein Alk is a C1 to C6 alkyl, and Ar is an aromatic ring. A non-limiting example of an alkaryl is benzyl.

Acyl means a carboxyl moiety comprising 1 to 4 carbons. Non-limiting examples include acetyl, propanoyl, butanoyl, or succinyl.

In an embodiment, Q is CR¹R² in Formula (I), thereby the cooling compound within the cooling composition is characterized by having a general Formula (II):

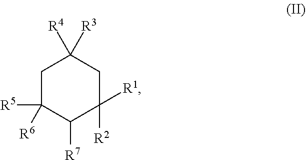

wherein R¹-R⁷ are the same as defined with respect to Formula (I).

In an embodiment, R⁴ is methyl and R⁶ is H in Formula (I), thereby the cooling compound within the cooling composition is characterized by having a general Formula (IIₐ):

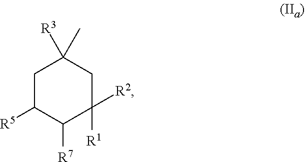

wherein R¹, R², R³, R⁵, and R⁷ are the same as defined with respect to Formula (I). In another embodiment, R¹ and R² are independently selected from the group consisting of CHO, CH₂OR¹¹, and CH₂OR¹²; R³ and R⁵ are independently selected from H and CH₃; R⁷ is selected from H and iso-propyl; and R¹¹ and R¹² are the same as defined with respect to Formula (I).

In an embodiment, R³ is CH₃; R⁴, R⁵, and R⁶ are each H; and R⁷ is isopropyl in Formula (II), thereby the cooling compound within the cooling composition is characterized by having a general Formula (III):

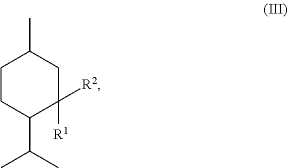

wherein R¹ and R² are the same as defined with respect to Formula (I). In another embodiment, R¹ and R² are independently selected from the group consisting of CHO, CH₂OR¹¹, and CH₂OR¹²; wherein R¹¹ and R¹² are the same as defined with respect to Formula (I). For example, the cooling compound within the cooling composition may be a stereoisomer having a general Formula (IVₐ) or (IV_{b}):

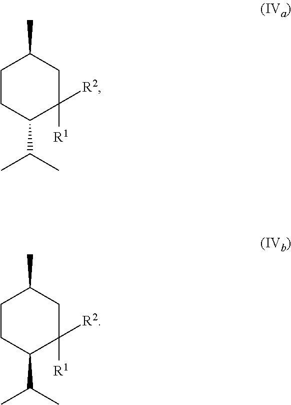

In an embodiment, R¹ and R² are each CHO in Formulas (IVₐ) and (IV_{b}), thereby providing compound (2S,5R)-2-isopropyl-5-methylcyclohexane-1,1-dicarbaldehyde and compound (2R,5R)-2-isopropyl-5-methylcyclohexane-1,1-dicarbaldehyde, respectively.

In another embodiment, R¹ is CHR¹⁰OR¹¹, and R² is CHR¹⁰OR¹² in Formulas (III), (IVₐ), or Formula (IV_{b}), thereby the cooling compound within the cooling composition is characterized by having a general Formula (V), (Vₐ) or (V_{b}):

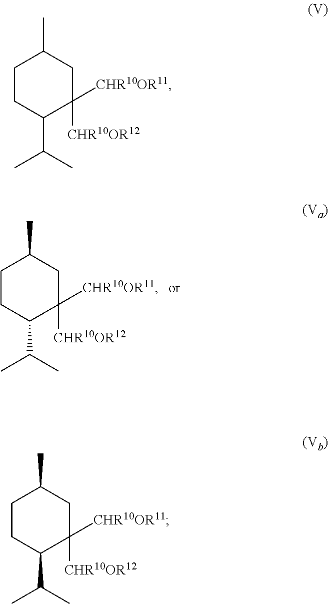

where R¹⁰, R¹¹, and R¹² are the same as defined with respect to Formula (I). In another embodiment, R¹⁰ is H in Formula (V), (Vₐ), or (V_{b}). In a further embodiment R¹⁰ is H, and R¹¹ and R¹² in combination form a spiroketal or a spiroacetal moiety CR¹³R¹⁴, thereby providing the cooling compound within the cooling composition is characterized by having a general Formula (VI), (VIₐ), or (VI_{b}):

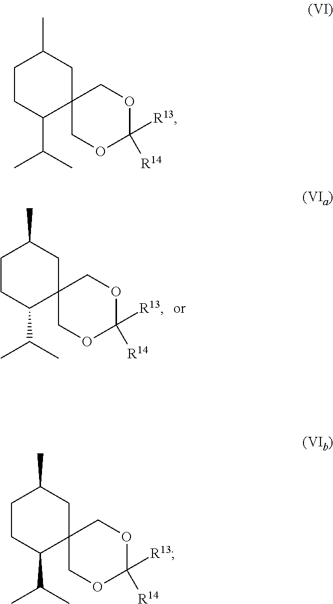

wherein R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls; or R¹³ and R¹⁴ in combination form a substituted or unsubstituted cycloalkyl.

In another embodiment, Q is CH-CH(CH₂OR⁸) CH₂OR⁹ in Formula (I), thereby the cooling compound within the cooling composition is characterized by having a general Formula (VII):

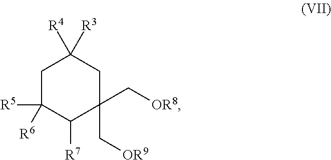

wherein R³ to R⁹ are the same as defined with respect to Formula (I).

In yet another embodiment, R¹ is CHR¹⁰OR¹¹, R² is CHR¹⁰R¹², and R¹⁰ is H in Formula (II), thereby the cooling compound within the cooling composition is characterized by having a general Formula (VIII):

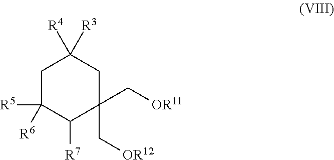

where R³ to R⁹, R¹¹, and R¹² are the same as defined with respect to Formula (I). In a further embodiment, R¹¹ and R¹² in combination form a spiroketal or a spiroacetal moiety CR¹³R¹⁴, thereby the cooling compound within the cooling composition is characterized by having a general Formula (IX):

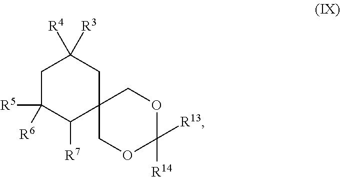

where R¹³ and R¹⁴ are the same as defined with respect to Formula (VI), (VIₐ), and (VI_{b}).

In a further embodiment, R³, R⁴, and R⁵ are each CH₃, and R⁶ and R⁷ are each H in Formula (II), thereby the cooling compound within the cooling composition is characterized by having a general Formula (X):

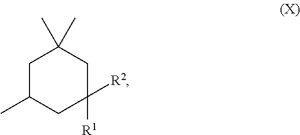

wherein R¹ and R² are the same as defined with respect to Formula (I). In another embodiment, R¹ and R² are independently selected from the group consisting of CHO, CH₂OR¹¹, and CH₂OR¹²; and wherein R¹¹ and R¹² are the same as defined with respect to Formula (I).

In another embodiment, R¹ is CHR¹⁰OR¹¹, R² is CHR¹⁰R¹², and R¹⁰ is H in Formula (X), thereby the cooling compound within the cooling composition is characterized by having a general Formula (VII);

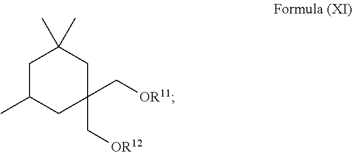

wherein R¹¹ and R¹² are the same as defined with respect to Formula (I). In a further embodiment, R¹¹ and R¹² in combination form a spiroketal or a spiroacetal moiety CR¹³R¹⁴, thereby the cooling compound within the cooling composition is characterized by having a general Formula (XII):

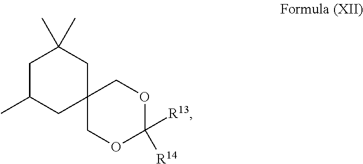

wherein R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls; or R¹³ and R¹⁴ in combination form a substituted or unsubstituted cycloalkyl.

According to an embodiment, the cooling composition comprises a cooling compound selected from the group consisting of:
1. ((2S,5R)-2-isopropyl-5-methylcyclohexane-1,1-diyl)dimethanol;
2. (7S,1OR)-7-isopropyl-3,10-dimethyl-2,4-dioxaspiro[5.5]undecane;
3. (7S,1OR)-7-isopropyl-3,3,10-trimethyl-2,4-dioxaspiro[5.5]undecane;
4. (7S,1OR)-3-ethyl-7-isopropyl-10-methyl-2,4-dioxaspiro[5.5]undecane;
5. 4-((7S,10R)-7-isopropyl-10-methyl-2,4-dioxaspiro[5.5]undecan-3-yl)-2-methoxyphenol;
6. (7S,1OR)-7-isopropyl-10-methyl-2,4-dioxaspiro[5.5]undecane;
7. (1S,4R,10S,13R)-1,10-diisopropyl-4,13-dimethyl-7,16-dioxadispiro[5.2.5⁹0.2⁶]hexadecane;
8. (2S,5R)-2-isopropyl-5-methylcyclohexane-1,1-dicarbaldehyde;
9. ((2S,5R)-2-isopropyl-5-methylcyclohexane-1,1-diyl)bis(methylene) diacetate;
10. (3,3,5-trimethylcyclohexane-1,1-diyl)dimethanol;
11. 3,8,8,10-tetramethyl-2,4-dioxaspiro[5.5]undecane;
12. 3,3,8,8,10-pentamethyl-2,4-dioxaspiro[5.5]undecane;
13. (7S,1OR)-3-ethyl-7-isopropyl-3,10-dimethyl-2,4-dioxaspiro[5.5]undecane;
14. (7S,1OR)-7-isopropyl-10-methyl-3-propyl-2,4-dioxaspiro[5.5]undecane;
15. (7S,1OR)-3,7-diisopropyl-10-methyl-2,4-dioxaspiro[5.5]undecane;
16. (7S,1OR)-3-(sec-butyl)-7-isopropyl-10-methyl-2,4-dioxaspiro[5.5]undecane;
17. (2S,5R)-2-isopropyl-1,1-bis(methoxymethyl)-5-methylcyclohexane;
18. (7S,1OR)-7-isopropyl-10-methyl-3-phenyl-2,4-dioxaspiro[5.5]undecane;
19. (7S,1OR)-3,3-diethyl-7-isopropyl-10-methyl-2,4-dioxaspiro[5.5]undecane;
20. (3,3-dimethylcyclohexane-1,1-diyl)dimethanol;
21. 4,4′-((((2S,5R)-2-isopropyl-5-methylcyclohexane-1,1-diyl)bis(methylene)) bis(oxy))bis(4-oxobutanoic acid);
22. (7S,1OR)-7-isopropyl-3,10-dimethyl-3-phenyl-2,4-dioxaspiro[5.5]undecane;
23. 2-((7S,10R)-7-isopropyl-10-methyl-2,4-dioxaspiro[5.5]undecan-3-yl)pyridine;
24. (7S,1OR)-7-isopropyl-10-methyl-3-(thiophen-2-yl)-2,4-dioxaspiro[5.5]undecane;
25. (7S,1OR)-7-isopropyl-10-methyl-3-(2-(methylthio)ethyl)-2,4-dioxaspiro[5.5]undecane;
26. 3,8,8-trimethyl-2,4-dioxaspiro[5.5]undecane;
27. (2-(sec-butyl)cyclohexane-1,1-diyl)dimethanol;
28. Ethyl (7S,1OR)-7-isopropyl-10-methyl-2,4-dioxaspiro[5.5]undecane-3-carboxylate;
29. 2,2′-((2S,5R)-2-isopropyl-5-methylcyclohexane-1,1-diyl)bis(ethan-1-ol);
30. (2-isopropyl-5,5-dimethylcyclohexane-1,1-diyl)dimethanol;
31. 7-(sec-butyl)-3-methyl-2,4-dioxaspiro[5.5]undecane;
32. ((2S,5R)-1-(hydroxymethyl)-2-isopropyl-5-methylcyclohexyl)methyl acetate;
33. 7-isopropyl-3,10,10-trimethyl-2,4-dioxaspiro[5.5]undecane;
34. 1,1′-((2S,5R)-2-isopropyl-5-methylcyclohexane-1,1-diyl)bis(ethan-1-ol);
35. 2-((1R,2R,5R)-5-methyl-2-(prop-1-en-2-yl)cyclohexyl)propane-1,3-diol;
36. 2-methyl-5-((1R,2R,5R)-5-methyl-2-(prop-1-en-2-yl)cyclohexyl)-1,3-dioxane;
37. 2-((1R,2S,5R)-2-isopropyl-5-methylcyclohexyl)propane-1,3-diol;
38. ((2S,5R)-1-((benzyloxy)methyl)-2-isopropyl-5-methylcyclohexyl)methanol;
39. (2S,5R)-1-(hydroxymethyl)-2-isopropyl-5-methylcyclohexane-1-carboxylic acid;
40. (3,3,5,5-tetramethylcyclohexane-1,1-diyl)dimethanol;
   as well as combinations and mixtures thereof.

In addition to the cooling compound having general Formulas (I) to (XII), the cooling composition further comprises an edible, potable, or cosmetic ingredient, such as those commonly used in ingestible or cosmetic products. For example, the edible, potable, or cosmetic ingredient may comprise a carrier material or diluent material, which may be inert or contain other active ingredients relevant to its end use. A wide variety of carrier or diluent materials can be employed including, for example, polar solvents, oils, fats, finely divided solids, maltodextrins, cyclodextrins, gums, natural or synthetic resins, or any other known carrier or diluent materials for cooling or flavoring compositions. In another example, the edible, potable, or cosmetic ingredient may comprise a flavorant, which may be particularly useful as a flavoring composition in a variety of ingestible (edible or potable) compositions and/or compositions destined for contact with the human or animal body (cosmetics). The flavorants may be selected from fruit flavors such as strawberry flavor, herbal oils such as eucalyptus oil, peppermint oil, spearmint oil, as well as other known flavors or flavoring oils which are conventionally employed in ingestible compositions, as well as those compositions designed for contact with human or animal bodies, such as flavoring syrups (e.g., sorbitol syrup) or other sweetening syrups.

The cooling composition or the flavorants may be diluted with a polar solvent such as, for example, ethyl alcohol, ethyl acetate, propylene glycol, isopropyl alcohol, glycerin, and combinations thereof. The polar solvent functions as a carrier material, which aids in incorporating the cooling composition into a product. The edible, potable, or cosmetic ingredient may optionally comprise one or more additional conventional components selected from the group consisting of colorants, lubricants, thickeners, emulsifiers, plasticizers, and encapsulating agents such as gums, starches, dextrins, and cyclodextrins, for example.

Typically the cooling composition will include 1 wt % to 99 wt % of the cooling compound, based on the entire weight of the cooling composition. Preferably, the cooling composition comprises 5 wt % to 90 wt % of the cooling compound, and 10 wt % to 95 wt % of the edible, potable, or cosmetic ingredient. More preferably, the cooling composition comprises 5 wt % to 50 wt % of the cooling compound, and 50 wt % to 95 wt % of the edible, potable, or cosmetic ingredient.

Furthermore, embodiments of the present invention relate to a product composition selected from topical, ingestible, or tobacco products, such as oral care products, nasal care products, toiletries, filters, combustible papers and coating sheets for smoking tobacco products, chewing tobacco, chewing tobacco products, snuff tobacco products, chewing gum and chewing gum products, comprising:
a topical, ingestible, or tobacco base product, and
an effective amount of the cooling compound having the general Formula (I)-(XII).

In an embodiment, the cooling compounds of general Formulas (I)-(XII) may be used in low concentrations of up to 1 wt %, based on the total weight of the product composition into which they are incorporated, and provide a pleasing cooling, or long-lasting cooling effect without the bitterness expected from many cooling compounds of the prior art. Further, at concentrations of up to 1 wt %, the cooling compounds do not develop a strong minty taste in the mouth or throat as do other cooling compounds or agents, such as menthol.

In accordance with an embodiment of the present invention, the topical products include, but are not limited to, toiletry products such as face creams, talcum powders, hair oils, shampoos, bath oils and salts, toilet soaps, cologne, antiperspirants, toilet water, perfume, shaving lotions and creams, soaps, creams, dentifrices, mouthwashes, lip balms, hair tonics, and other similar products.

In accordance with an embodiment of the present invention, the ingestible (e.g., edible, potable, etc.) products include, but are not limited to, alcoholic and non-alcoholic beverages, confectionery compositions including confectionery tablets, hard-boiled candies, chewing gums, chewing gum products, pectin-based candies, chewy candies, cream-centered candies and fondants; carbonated beverages, powdered beverage mixes, distilled beverages, mineral waters, baked goods, dairy products, fruit ices, jams, jellies, gelatins, puddings, and animal feeds. Furthermore, the cooling compounds of the present invention may enhance the taste sensation of alcohol in alcoholic beverages. As a result, alcoholic beverages comprising the cooling compounds of the present invention may taste like they have a higher alcohol content than equally strong alcoholic beverages without said cooling compound.

In accordance with an embodiment of the present invention, the tobacco products include, but are not limited to, chewing tobacco products, snuff tobacco products, as well as filters, combustible papers, and coating sheets for smoking tobacco products.

The choice of the cooling compound within the scope of general Formulas (I)-(XII) for use in the cooling composition will depend, to some extent, on the solubility characteristics of the compound. For example, the cooling compounds of the present invention are sparingly soluble in water and more soluble in oil. Thus, the cooling compounds of the present invention are particularly suitable for environments where oil solubility is advantageous, although these cooling compounds can be used in aqueous environments if a concentration below the water solubility limit is employed.

The most preferred compositions incorporating the cooling compounds and/or cooling compounds of the present invention are pressed confectionery tablets, hard-boiled candies, chewing gums, chewy candies, pectin candies, cream-centered candies, fondant, toothpastes, mouthwashes, breath fresheners, alcoholic and non-alcoholic beverages, carbonated beverages, and dry beverage mixes.

The amount of cooling compound or cooling composition incorporated in each of these proposed product compositions (or end use compositions) will vary depending upon the particular compound, concentration of the cooling compounds in the cooling composition, the degree of cooling effect desired, and the strength of other flavorants in the composition, etc. Typically, the cooling compound having a general Formula (I)-(XII) will make up from 0.001-1.0% by weight of the end use composition. More preferably, the cooling compound makes up 0.005 to 0.5% by weight, based on the total weight of the end use composition.

It has been found that the cooling compounds of the present invention provide a cooling effect in a different area of the mouth and throat when ingested than, for example, menthol or carboxamide-based coolant agents. As a result, the cooling compounds of the present invention further provide a complementary or synergistic cooling effect when combined with at least one secondary coolant agent. Exemplary, but non-limiting, secondary coolant compounds include carboxamides, ketals, menthyl glutarate, menthyl succinate, menthyl acetate, menthyl lactate, 3-menthoxypropane-1,2 diol, isopulegol, menthol, and mixtures thereof. Thus, in accordance with another embodiment of the present invention, a cooling composition is provided comprising the cooling compound having a general Formula (I)-(XII), in combination with at least one secondary coolant agent. Secondary coolant agents which may be used in combination with the cooling compounds of the present invention include carboxamides, ketals, menthyl glutarate, menthyl succinate, menthyl acetate, menthyl lactate, 3-menthoxypropane-1,2 diol, menthol, and mixtures thereof.

Carboxamide and ketal coolant agents are known from the prior art and are described, for example, in U.S. Pat. No. 5,009,893 and International Patent Application Publ. No. WO-93/23005, the disclosures of which are hereby incorporated by reference. The remaining secondary coolant agents are known cooling agents, many of which are commercially available.

For example, the secondary carboxamide coolant agent may be selected from N-substituted-p-menthane-3-carboxamides of the general formula:

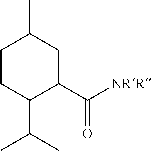

wherein R′, when taken separately, is hydrogen or an aliphatic radical containing up to 25 carbon atoms; R″ when taken separately is hydroxy or an aliphatic radical containing up to 25 carbon atoms, with the proviso that when R′ is hydrogen R″ may also be an aryl radical of up to 10 carbon atoms and selected from the group consisting of substituted phenyl, phenylalkyl, substituted phenylalkyl, naphthyl, substituted naphthyl and pyridyl; and R′ and R″, when taken together with the nitrogen atom to which they are attached, represent a cyclic or heterocylic group of up to 25 carbon atoms.

Additionally, the secondary carboxamide coolant agent may be selected from acyclic tertiary and secondary carboxamides of the general formula:

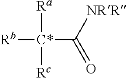

where R′ and R″ are independently selected from hydrogen, C1-C5 alkyl or C1-C8 hydroxyalkyl and provide a total of no more than 8 carbon atoms, with the proviso that when R′ is hydrogen, R″ may also be alkylcarboxyalkyl of up to 6 carbon atoms; or wherein R and R″, when taken together, represent an alkylene group of up to 6 carbon atoms, the opposite ends of which group are attached to the amide nitrogen atom thereby to form a nitrogen heterocycle, the carbon chain of which may optionally be interrupted by oxygen; R^{a} is hydrogen or C1-C5 alkyl; and R^{b} and R^{c} are each C1-C5 alkyl; with the provisos that (i) R^{a}, R^{b}, and R^{c} together provide a total of at least 5 carbon atoms, preferably from 5-10 carbon atoms; and (ii) when R^{a} is hydrogen, R^{b} is C2-C5 alkyl and R^{c} is C2-C5 alkyl and at least one of R^{b} and R^{c} is branched, preferably in an alpha or beta position relative to the carbon atom marked (*) in the formula; and mixtures thereof.

Non-limiting secondary ketal coolant agents may be represented by the general formula:

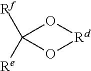

in which R^{d} represents a C2-C6 alkylene radical having at least 1, but not more than 3, hydroxyl group(s), and either R^{e} and R^{f} independently of one another represent C1-C10 alkyls which are optionally substituted by 1 to 3 radicals selected from the group consisting of hydroxyl, amino, and halogen; C5-C7 cycloalkyls, preferably cyclohexyl; and C6-C12 aryls, preferably phenyl, with the proviso that the total of the carbon atoms of R^{e} and R^{f} is not less than 3, or wherein R^{e} and R^{f} together represent an alkylene radical which, together with the carbon atom which carries the radicals R^{e} and R^{f}, forms a 5-7 membered ring, optionally substituted by C1-C6 alkyl group(s).

Depending upon the particular flavor desired, the relative amounts of the cooling compound of Formulas (I)-(XII) vis-à-vis the secondary coolant agents in the cooling composition may be varied over a wide range. For example, when a strong minty taste of menthol is desirable, a combination of a larger quantity of menthol with a relatively small quantity of the cooling compound of the present invention may be desirable. Other potential combinations of the inventive cooling compounds with secondary coolant agents will be apparent to the man of skill in the art.

When present, the level of the secondary coolant agent in the cooling composition is from about 0.05% by weight to about 95% by weight, more preferably from about 0.1% by weight to about 70% by weight, and most preferably from about 0.5% by weight to about 50% by weight, based on the total weight of the cooling composition. Typically, the cooling compositions are made by mixing the cooling compounds having a general Formula (I)-(XII) and secondary coolant agents together in a conventional manner.

In accordance with yet another embodiment of the present invention, a cooling compound is provided having a general Formula (I):

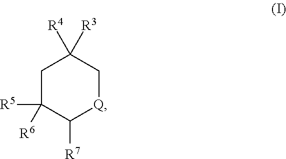

wherein Q is selected from CR¹R² or CH-CH(CH₂OR⁸)CH₂OR⁹; wherein R¹ is selected from the group consisting of CHO, CO₂H; CH₂CH₂OH; CHR¹⁰R¹¹; wherein R² is selected from the group consisting of CHO, CH₂CH₂OH, and CHR¹⁰OR¹²; wherein R⁸ and R⁹ are independently H, or in combination form a spiroacetal or spiroketal moiety; wherein R¹⁰ is H or CH₃; wherein R¹¹ and R¹² are independently selected from H, substituted or unsubstituted C1 to C6 alkyls, substituted or unsubstituted cycloalkyls, substituted or unsubstituted alkaryls, substituted or unsubstituted aryls or heteroaryls, and substituted or unsubstituted acyls, or wherein R¹¹ and R¹² in combination form a spiroketal or a spiroacetal moiety; and
wherein R³, R⁴, R⁵, and R⁶ are independently selected from H, CH₃, and CH₂CH₃; where R⁷ is selected from iso-propyl, isopropenyl, and sec-butyl;
   with a first proviso that R³, R⁴, R⁵, R⁶, and R⁷ are not all H,
   with a second proviso that when Q is CR¹R², and R¹ and R² are both CH₂OH:
      then R⁷ is not H; and
      then R³, R⁵, and R⁶ are not all H, R⁴ is not CH₃, and R⁷ is not isopropyl;
   with a third proviso that when Q is CR¹R², R¹ and R² are both CH₂OH, and R⁷ is isopropyl or isobutyl, then at least two or more of R³, R⁴, R⁵, and R⁶ are CH₃ or CH₂CH₃; and
   with a fourth proviso that when Q is CR¹R², and R¹ is CH₂R¹⁰R¹², R² is CH₂R¹⁰R¹², R¹⁰ is H, and R¹¹ and R¹² in combination form a spiroketal or a spiroacetal moiety, and R⁷ is H, then at least two or more of R³, R⁴, R⁵, and R⁶ are CH₃ or CH₂CH₃.

Use of the cooling compound having a general Formula (I) as a cooling agent or a perfume agent is further contemplated.

In accordance with one or more embodiments, the cooling compounds themselves may be further defined by Formulas (II)-(XII), wherein Q and R¹ to R¹⁴ are defined in said Formulas (II)-(XII), being only limited by the foregoing four provisos applicable thereto.

Exemplary compounds and compositions within embodiments of the present invention are illustrated by the following examples.

### EXPERIMENTAL PROCEDURE FOR THE EXAMPLES: Synthesis of the Cooling Compounds 1 to 40

### Compound 1: ((2S,5R)-2-isopropyl-5-methylcyclohexane-1,1-diyl)dimethanol

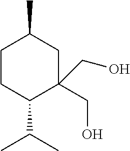

In a 1 L flask are added potassium hydroxide (80 g, 1426 mmol) and ethanol (500 ml). After complete solubilization of KOH, 37% aqueous formaldehyde (106 ml, 1426 mmol) is added to the medium over 15 min and the mixture is stirred for 30 min at room temperature. (2S, 5R)-2-isopropyl-5-methylcyclohexane-1-carbaldehyde (60 g, 357 mmol) (also called menthanal, the synthesis of which is described in the literature) is then added to the medium over 15 min then the mixture is stirred 72 h at room temperature. The medium is concentrated and the aqueous phase extracted with MTBE (300 mL). The organic phases are washed with a saturated solution of NaHCO₃ (150 mL), and the aqueous phase is extracted with methyl t-butyl ether (MTBE) (150 mL). The organic phases are combined and washed with brine (100 mL), dried over anhydrous MgSO₄, filtered, and concentrated. ((2S, 5R)-2-isopropyl-5-methylcyclohexane-1,1-diyl) dimethanol (45.93 g, 218 mmol, 61.1% yield) is obtained after distillation (T=122° C., 0.3 mbar) in the form of a very viscous liquid which crystallized into a white solid after a few hours at room temperature. ¹H NMR (300 MHz, Chloroform-d) δ 4.07 (d, J=10.4 Hz, 1H), 3.87 (d, J=1.0 Hz, 2H), 3.39 (d, J=10.4 Hz, 1H), 2.69 (d, J=1.8 Hz, 2H), 2.27-2.15 (m, 1H), 2.05-1.90 (m, 1H), 1.85-1.71 (m, 1H), 1.67-1.37 (m, 2H), 1.34-1.15 (m, 1H), 1.08 (dd, J=12.8, 3.5, 1.2 Hz, 1H), 0.94-0.85 (m, 6H), 0.87-0.76 (m, 1H), 0.74 (d, J=6.8 Hz, 3H), 0.70-0.55 (m, 1H). ¹³C NMR, IR (cm⁻¹) and Mass Spectrometry data were consistent with the expected data for the obtained compound. Olfactive description: Almost odorless, fresh.

### Compound 2: (7S,1OR)-7-isopropyl-3,10-dimethyl-2,4-dioxaspiro[5.5]undecane

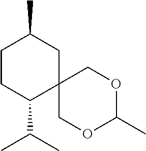 [SMILES: CC1OCC2(CO1)C[C@H](C)CC[C@H]2C(C)C]

In a 100 mL flask fitted with magnetic stirring and a condenser are added ((2S, 5R)-2-isopropyl-5-methylcyclohexane-1,1-diyl) dimethanol (5 g, 24.96 mmol), acetaldehyde (2.81 ml, 49.9 mmol), and para-toluene sulfonic acid monohydrate (PTSA) (0.750 g, 3.94 mmol). The medium is left under stirring at ambient temperature for 1 night. Na₂CO₃ (0.579 g, 5 mol %) is added to the medium which is then filtered. (7S, 1OR)-7-isopropyl-3,10-dimethyl-2,4-dioxaspiro [5.5]undecane (1.3 g, 23%) is obtained as a colorless liquid after distillation (70° C., 0.3 mbar; mixture of isomers, 2 GC peaks 25/75). ¹H NMR (300 MHz, CDCl₃) δ 4.61 (q, J=5.2 Hz, 1H), 4.09-3.97 (m, 2H), 3.79-3.68 (m, 1H), 3.51-3.31 (m, 1H), 2.65-2.53 (m, 1H), 2.12-1.93 (m, 1H), 1.84-1.70 (m, 1H), 1.63-1.51 (m, 1H), 1.55-1.42 (m, 2H), 1.38-1.20 (m, 3H), 1.25-1.06 (m, 1H), 1.01-0.82 (m, 7H), 0.84-0.71 (m, 3H), 0.71-0.49 (m, 1H). ¹³C NMR, IR (cm⁻¹) and Mass Spectrometry data were consistent with the expected data for the obtained compound. Olfactive description: Fresh, minty, fruity.

### Compound 3: (7S,1OR)-7-isopropyl-3,3,10-trimethyl-2,4-dioxaspiro[5.5]undecane

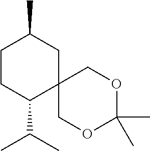 [SMILES: CC(C)[C@@H]1CC[C@@H](C)CC12COC(C)(C)OC2]

In a 100 mL flask fitted with magnetic stirring and a condenser are added ((2S, 5R)-2-isopropyl-5-methylcyclohexane-1,1-diyl) dimethanol (10 g, 49.9 mmol) melt, acetone (5.80 g, 100 mmol), and PTSA (0.475 g, 2.496 mmol). The medium is left under stirring at ambient temperature for 1 night. Na₂CO₃ (0.442 g, 5 mol %) is added to the medium which is then filtered and concentrated. The liquid obtained is purified by chromatography column (CombiFlash®, cyclohexane/EtOAc gradient). (7S, 1OR)-7-isopropyl-3,3,10-trimethyl-2,4-dioxaspiro [5.5]undecane (1.81 g, 14%) is obtained as a colorless liquid. ¹H NMR (300 MHz, Chloroform-d) δ 3.89 (d, J=11.6 Hz, 2H), 3.60 (dt, J=11.7, 0.9 Hz, 1H), 3.20-3.10 (m, 1H), 2.29-2.08 (m, 2H), 1.75-1.60 (m, 1H), 1.49-1.37 (m, 1H), 1.33 (s, 3H), 1.32 (s, 3H), 1.23-1.00 (m, 1H), 0.99-0.60 (m, 11H), 0.58-0.42 (m, 1H). ¹³C NMR, IR (cm⁻¹) and Mass Spectrometry data were consistent with the expected data for the obtained compound. Olfactive description: Weak, fresh, woody.

### Compound 4: (7S,1OR)-3-ethyl-7-isopropyl-10-methyl-2,4-dioxaspiro[5.5]undecane

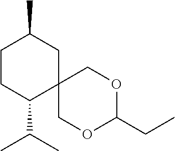 [SMILES: CCC1OCC2(CO1)C[C@H](C)CC[C@H]2C(C)C]

In a 100 mL flask fitted with magnetic stirring and a condenser are added ((2S, 5R)-2-isopropyl-5-methylcyclohexane-1,1-diyl) dimethanol (7 g, 34.9 mmol), propionaldehyde (5.01 mL, 69.9 mmol), and PTSA (0.332 g, 1.747 mmol). The medium is left under stirring at ambient temperature for 1 night. Na₂CO₃ (0.185 g, 5 mol %) is added to the medium which is then filtered and concentrated. (7S, 1OR)-3-ethyl-7-isopropyl-10-methyl-2,4-dioxaspiro [5.5]undecane (3.86 g, 44%) is obtained as a colorless liquid after distillation (T=80-85° C., 0.3 mbar; 2 peaks GC 23/77). ¹H NMR (300 MHz, Chloroform-d) δ 4.44-4.29 (m, 1H), 4.10-3.96 (m, 2H), 3.80-3.68 (m, 1H), 3.50-3.32 (m, 1H), 2.57 (ddd, J=13.6, 3.4, 2.3 Hz, 1H), 2.13-1.93 (m, 1H), 1.83-1.71 (m, 1H), 1.70-1.58 (m, 2H), 1.57-1.41 (m, 1H), 1.38-1.20 (m, 1H), 1.20-1.03 (m, 1H), 1.01-0.83 (m, 11H), 0.78 (d, J=6.9 Hz, 3H), 0.67-0.49 (m, 1H). ¹³C NMR; IR (cm⁻¹); and Mass Spectrometry data were consistent with the expected data for the obtained compound. Olfactive description: Fresh, fruity, exotic.

### Compound 5: 4-((7S,10R)-7-isopropyl-10-methyl-2,4-dioxaspiro[5.5]undecan-3-yl)-2-methoxyphenol

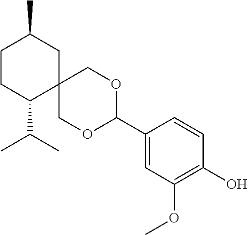 [SMILES: COc1cc(C2OCC3(CO2)C[C@H](C)CC[C@H]3C(C)C)ccc1O]

In a 100 mL flask fitted with magnetic stirring and a condenser are added vanillin (1.246 g, 8.19 mmol), trimethyl orthoformate (2.73 mL, 24.96 mmol), PTSA (0.047 g, 0.250 mmol), and then ((2S, 5R)-2-isopropyl-5-methylcyclohexane-1,1-diyl) dimethanol (1 g, 4.99 mmol). The medium is left under stirring at ambient temperature overnight. MTBE (50 mL) is added then the medium is poured into a saturated NaHCO₃ (50 mL) solution. The organic phase is washed with NaCl (sat) (100 mL), dried over anhydrous MgSO₄, filtered, and concentrated. The liquid obtained is purified by chromatography column (CombiFlash®, cyclohexane/EtOAc gradient). 4-((7S, 1OR)-7-isopropyl-10-methyl-2,4-dioxaspiro [5.5]undecan-3-yl)-2-methoxyphenol (0.53 g, 31%) is obtained as a colorless viscous liquid. ¹H NMR (300 MHz, Chloroform-d) δ 7.10-6.91 (m, 2H), 6.94-6.84 (m, 1H), 5.58 (s, 1H), 5.36 (d, J=10.7 Hz, 1H), 4.27-4.16 (m, 2H), 4.01-3.91 (m, 1H), 3.87 (s, 3H), 3.73-3.50 (m, 1H), 2.81-2.69 (m, 1H), 2.21-2.01 (m, 1H), 1.81 (dt, J=12.4, 3.1 Hz, 1H), 1.71-1.46 (m, 2H), 1.45-1.10 (m, 2H), 1.08-0.98 (m, 2H), 0.98-0.94 (m, 4H), 0.94-0.80 (m, 4H), 0.76-0.55 (m, 1H). ¹³C NMR; IR (cm⁻¹); and Mass Spectrometry data were consistent with the expected data for the obtained compound. Olfactive description: Almost odorless, vanilla.

### Compound 6: (7S,1OR)-7-isopropyl-10-methyl-2,4-dioxaspiro[5.5]undecane

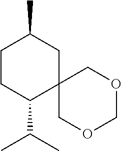

In a 250 mL three-necked flask fitted with a Dean-Stark trap are added ((2S, 5R)-2-isopropyl-5-methylcyclohexane-1,1-diyl) dimethanol (10 g, 49.9 mmol), dimethoxymethane (22.34 ml, 250 mmol), cyclohexane (100 ml) and PTSA (0.475 g, 2.496 mmol). The mixture is stirred at reflux overnight. The cooled medium is poured into a saturated NaHCO₃ (50 mL) solution, then the organic phase is washed with brine (50 mL), dried over anhydrous MgSO₄, filtered, and concentrated. (7S, 1OR)-7-isopropyl-10-methyl-2,4-dioxaspiro [5.5]undecane (7.13 g, 32.9 mmol, 65.9% yield) is recovered as a colorless liquid after distillation (74° C., 0.4 mbar). ¹H NMR (300 MHz, Chloroform-d) δ 4.93 (d, J=5.9 Hz, 1H), 4.67 (d, J=6.0 Hz, 1H), 3.97-3.87 (m, 2H), 3.82 (dd, J=11.4, 1.3 Hz, 1H), 3.45-3.29 (m, 1H), 2.43-2.28 (m, 1H), 2.20 (p, J=6.9 Hz, 1H), 1.88-1.64 (m, 1H), 1.64-1.36 (m, 2H), 1.19 (qd, J=13.1, 3.5 Hz, 1H), 0.96 (dd, J=3.6, 1.2 Hz, 1H), 0.91 (d, J=5.0 Hz, 3H), 0.89 (d, J=4.3 Hz, 3H), 0.85 (dd, J=3.6, 1.0 Hz, 1H), 0.80 (d, J=6.7 Hz, 3H), 0.67-0.53 (m, 1H). ¹³C NMR; IR (cm⁻¹); and Mass Spectrometry data were consistent with the expected data for the obtained compound. Olfactive description: Weak, red fruits.

### Compound 7: (1S,4R,10S,13R)-1,10-diisopropyl-4,13-dimethyl-7,16-dioxadispiro [5.2.5⁹0.2⁶]hexadecane

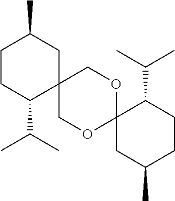 [SMILES: CC(C)[C@@H]1CC[C@@H](C)CC12COC1(C[C@H](C)CC[C@H]1C(C)C)OC2]

In a 100 mL three-necked flask fitted with a Dean-Stark trap are added ((2S, 5R)-2-isopropyl-5-methylcyclohexane-1,1-diyl) dimethanol (2 g, 9.98 mmol), l-menthone (1.540 g, 9.98 mmol), toluene (50 ml), and then PTSA (0.095 g, 0.499 mmol). The mixture is stirred at reflux overnight. The cooled medium is poured into a saturated NaHCO₃ (50 mL) solution, and then the organic phase is washed with brine (50 mL), dried over anhydrous MgSO₄, filtered, and concentrated. (1S, 4R, 10S, 13R)-1,10-diisopropyl-4,13-dimethyl-7,16-dioxadispiro [5.2.5⁹0.2⁶]hexadecane (1.5 g, 4.23 mmol, 42.4% yield) is recovered in the form of a pasty white solid after purification by chromatographic column (CombiFlash®, cyclohexane/EtOAc gradient). ¹H NMR (300 MHz, Chloroform-d) δ 4.35 (d, J=11.1 Hz, 1H), 3.89-3.67 (m, 2H), 3.07 (dd, J=11.1, 2.6 Hz, 1H), 2.79-2.63 (m, 2H), 2.63-2.47 (m, 1H), 2.14-1.89 (m, 1H), 1.83-1.66 (m, 3H), 1.58-1.47 (m, 4H), 1.28-1.06 (m, 3H), 0.95-0.87 (m, 18H), 0.81 (d, J=6.8 Hz, 4H). ¹³C NMR; IR (cm⁻¹); and Mass Spectrometry data were consistent with the expected data for the obtained compound. Olfactive description: Odorless.

### Compound 8: (2S,5R)-2-isopropyl-5-methylcyclohexane-1,1-dicarbaldehyde

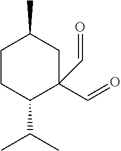

In a 500 mL three-necked flask are added TEMPO (0.234 g, 1.498 mmol), ((2S, 5R)-2-isopropyl-5-methylcyclohexane-1,1-diyl) dimethanol (15 g, 74.9 mmol), ethyl acetate (100 mL), and sodium bicarbonate (12.58 g, 150 mmol). A solution of potassium bromide (1.782 g, 14.98 mmol) in water (20.00 ml) is then added to the flask. The mixture is stirred vigorously and cooled to 0-5° C. 36° bleach (116 ml, 187 mmol) is then added dropwise over 20 min while maintaining the temperature below 15° C., then the mixture is stirred for 30 min in an ice bath. The medium is diluted with water (50 mL) and the aqueous phase is extracted with EtOAc (100 mL). The organic phases are combined and then washed with a 10% aqueous H₂SO₄ (30 mL) solution, and a saturated Na₂SO₃ (50 mL) solution. No residual oxidant is observed in the organic phase with a KI/Starch test. The organic phase is washed with sat. NaCl (50 mL), dried over anhydrous MgSO₄, filtered, and concentrated. (2S, 5R)-2-isopropyl-5-methylcyclohexane-1,1-dicarbaldehyde (1.5 g, 7.11 mmol, 9.49% yield) is obtained as a yellowish liquid with GC purity of 93% after distillation (T=78-80° C., 0.4 mbar). ¹H NMR (300 MHz, Chloroform-d) δ 10.12 (t, J=0.9 Hz, 1H), 9.64 (s, 1H), 1.96-1.77 (m, 4H), 1.75-1.58 (m, 2H), 1.55-1.34 (m, 1H), 1.12-0.95 (m, 2H), 0.93-0.88 (m, 9H). ¹³C NMR; IR (cm⁻¹); and Mass Spectrometry data were consistent with the expected data for the obtained compound. Olfactive description: Fresh, minty.

### Compound 9: ((2S,5R)-2-isopropyl-5-methylcyclohexane-1,1-diyl)bis(methylene) diacetate

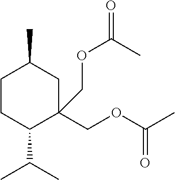 [SMILES: CC(=O)OCC1(COC(C)=O)C[C@H](C)CC[C@H]1C(C)C]

In a 100 mL three-necked flask fitted with a condenser are added ((2S, 5R)-2-isopropyl-5-methylcyclohexane-1,1-diyl) dimethanol (10 g, 49.9 mmol), MTBE (30 ml), 4-dimethylaminopyridine (DMAP) (0.610 g, 4.99 mmol), and then acetic anhydride (14.13 ml, 150 mmol) is added dropwise. The mixture is stirred at room temperature for 30 min. The medium is poured into a mixture of 10% aqueous HCl solution (30 mL) and ice, then the organic phase is washed with a saturated NaHCO₃ (30 mL) solution, sat. NaCl (30 mL), dried over anhydrous MgSO₄, filtered, and concentrated. ((2S, 5R)-2-isopropyl-5-methylcyclohexane-1,1-diyl)bis(methylene) diacetate (11.2 g, 38.2 mmol, 77% yield) is recovered as a clear colorless liquid after distillation (T=106-108° C., 0.7 mbar). ¹H NMR (300 MHz, Chloroform-d) δ 4.26 (d, J=11.4 Hz, 1H), 4.08-3.97 (m, 3H), 2.06 (s, 3H), 2.03 (s, 3H), 1.99-1.89 (m, 1H), 1.83-1.58 (m, 2H), 1.58-1.42 (m, 2H), 1.42-1.11 (m, 2H), 1.01-0.90 (m, 1H), 0.88 (d, J=6.9 Hz, 3H), 0.84 (d, J=6.4 Hz, 3H), 0.82-0.78 (m, 1H), 0.75 (d, J=6.8 Hz, 3H). ¹³C NMR; IR (cm⁻¹); and Mass Spectrometry data were consistent with the expected data for the obtained compound. Olfactive description: Odorless.

### Compound 10: (3,3,5-trimethylcyclohexane-1,1-diyl)dimethanol

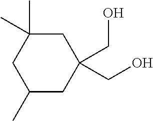

In a 250 ml three-necked flask are added potassium hydroxide (21.82 g, 389 mmol) and ethanol (162 ml). After complete solubilization of the KOH, 37% aqueous formaldehyde (29.0 ml, 389 mmol) is added dropwise at ambient temperature, and then the mixture is stirred for 10 min. 3,3,5-trimethylcyclohexane-1-carbaldehyde (15 g, 97 mmol) is then added dropwise over 30 min and the mixture is stirred overnight at ambient temperature. The medium is concentrated and the aqueous phase extracted with MTBE (2×30 mL). The combined organic phases are washed sequentially with a saturated NaHCO₃ (30 mL) solution, and then brine (30 mL), dried over anhydrous MgSO₄, filtered, and concentrated to give a white solid. (3,3,5-trimethylcyclohexane-1,1-diyl) dimethanol (13.3 g, 67.8 mmol, 69.7% yield) is recovered in the form of a white solid after recrystallization from cyclohexane at reflux (50 mL). ¹H NMR (300 MHz, Chloroform-d) δ 3.85-3.66 (m, 2H), 3.45-3.31 (m, 2H), 3.31-3.01 (m, 2H), 1.72-1.46 (m, 3H), 1.41 (ddt, J=12.9, 3.6, 2.1 Hz, 1H), 0.98 (s, 3H), 0.92 (s, 3H), 0.88 (d, J=6.4 Hz, 3H), 0.86-0.67 (m, 2H), 0.59-0.46 (m, 1H). ¹³C NMR; IR (cm⁻¹); and Mass Spectrometry data were consistent with the expected data for the obtained compound. Olfactive description: Odorless.

### Compound 11: 3,8,8,10-tetramethyl-2,4-dioxaspiro[5.5]undecane

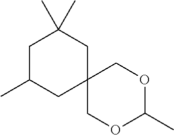

In a 100 mL three-necked flask are added (3,3,5-trimethylcyclohexane-1,1-diyl) dimethanol (8 g, 42.9 mmol), acetaldehyde (7.20 ml, 129 mmol) then PTSA (0.408 g, 2.147 mmol). The mixture is stirred at ambient temperature for 3 h, then sodium carbonate (0.455 g, 4.29 mmol) is added to the medium. After 5 min of stirring, the medium is filtered and then distilled to obtain 3,8,8,10-tetramethyl-2,4-dioxaspiro [5.5]undecane (3.34 g, 15.73 mmol, 36.6% yield) in the form of a colorless liquid (Tb.=54° C., 0.4 mbar; 2 GC peaks 60/40). ¹H NMR (300 MHz, Chloroform-d) δ 4.65-4.45 (m, 1H), 4.42-4.14 (m, 1H), 3.51-3.20 (m, 3H), 2.53-2.20 (m, 1H), 1.62-1.48 (m, 1H), 1.48-1.37 (m, 1H), 1.32 (t, J=5.3 Hz, 3H), 1.17-1.07 (m, 1H), 1.04 (s, 2H), 0.94 (s, 2H), 0.92 (d, J=6.4 Hz, 2H), 0.88-0.85 (m, 3H), 0.84 (s, 1H), 0.75 (ddd, J=19.2, 13.2, 6.6 Hz, 1H), 0.66-0.40 (m, 1H). ¹³C NMR; IR (cm⁻¹); and Mass Spectrometry data were consistent with the expected data for the obtained compound. Olfactive description: Woody, ambery, fruity.

### Compound 12: 3,3,8,8,10-pentamethyl-2,4-dioxaspiro[5.5]undecane

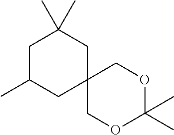

To a 50 mL two-necked flask are added (3,3,5-trimethylcyclohexane-1,1-diyl) dimethanol (5 g, 26.8 mmol), acetone (7.95 ml, 107 mmol), and PTSA (0.255 g, 1.342 mmol). The mixture is stirred for 1 h at room temperature. Sodium carbonate (0.284 g, 2.68 mmol) is added and the medium is stirred for 5 min before being filtered and then distilled. 3,3,8,8,10-pentamethyl-2,4-dioxaspiro [5.5]undecane (2.7 g, 11.81 mmol, 44.0% yield) is recovered in the form of a clear colorless liquid (T=58-62° C., 0.3 mbar). ¹H NMR (300 MHz, Chloroform-d) δ 3.92 (dd, J=11.7, 2.0 Hz, 1H), 3.72 (dd, J=11.6, 1.5 Hz, 1H), 3.52 (d, J=11.4 Hz, 1H), 3.23 (dd, J=11.4, 2.0 Hz, 1H), 2.24-2.11 (m, 1H), 1.81-1.53 (m, 1H), 1.40 (d, J=5.3 Hz, 7H), 1.31 (dt, J=13.9, 2.1 Hz, 1H), 0.95-0.86 (m, 9H), 0.82-0.67 (m, 2H), 0.61-0.44 (m, 1H). ¹³C NMR; IR (cm⁻¹); and Mass Spectrometry data were consistent with the expected data for the obtained compound. Olfactive description: Woody, fruity.

### Compound 13: (7S,1OR)-3-ethyl-7-isopropyl-3,10-dimethyl-2,4-dioxaspiro[5.5]undecane

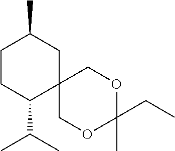 [SMILES: CCC1(C)OCC2(CO1)C[C@H](C)CC[C@H]2C(C)C]

To a 50 mL three-necked flask are added ((2S, 5R)-2-isopropyl-5-methylcyclohexane-1,1-diyl) dimethanol (10 g, 46.9 mmol), PTSA (0.446 g, 2.346 mmol), and butan-2-one (6.77 g, 94 mmol). The medium is stirred at ambient temperature overnight and then transferred to a 250 mL three-necked flask equipped with a Dean-Stark trap. Butan-2-one (6.77 g, 94 mmol) and cyclohexane (100 mL) are added and the mixture is refluxed for 1 h. Water (50 mL) is added and the aqueous phase is extracted with MTBE (2×30 mL). The organic phases are combined, washed with a saturated NaHCO₃ (40 mL) solution, brine (40 mL), dried over anhydrous MgSO₄, filtered, and concentrated. (7S, 1OR)-3-ethyl-7-isopropyl-3,10-dimethyl-2,4-dioxaspiro [5.5]undecane (5.8 g, 21.52 mmol, 45.9% yield) is obtained under the form of a colorless liquid (T=72-77° C., 0.4 mbar, 2 GC peaks 46/54). ¹H NMR (300 MHz, CDCl3) δ 4.06-3.83 (m, 2H), 3.66 (ddd, J=11.8, 7.0, 1.5 Hz, 1H), 3.29-3.08 (m, 1H), 2.34-2.05 (m, 2H), 1.83-1.57 (m, 3H), 1.57-1.40 (m, 2H), 1.33 (d, J=7.9 Hz, 3H), 1.28-1.02 (m, 1H), 0.99-0.80 (m, 14H), 0.64-0.46 (m, 1H). ¹³C NMR; IR (cm⁻¹); and Mass Spectrometry data were consistent with the expected data for the obtained compound. Olfactive description: Powerful, woody, algae.

### Compound 14: (7S,1OR)-7-isopropyl-10-methyl-3-propyl-2,4-dioxaspiro[5.5]undecane

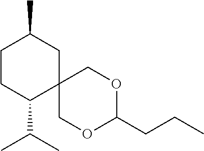 [SMILES: CCCC1OCC2(CO1)C[C@H](C)CC[C@H]2C(C)C]

To a 50 mL three-necked flask are added ((2S, 5R)-2-isopropyl-5-methylcyclohexane-1,1-diyl) dimethanol (10 g, 46.9 mmol), PTSA (0.446 g, 2.346 mmol), and butyraldehyde (13.53 g, 188 mmol). The medium is stirred at ambient temperature for 45 min then sodium carbonate (0.497 g, 4.69 mmol) is added. The mixture is stirred for 5 min, then filtered and distilled. (7S, 1OR)-7-isopropyl-10-methyl-3-propyl-2,4-dioxaspiro [5.5]undecane (9.6 g, 35.1 mmol, 74.8% yield) is recovered as a colorless liquid (T=66° C., 0.5 torr; 2 GC peaks 82/18). ¹H NMR (300 MHz, CDCl3) δ 4.44 (d, J=5.2 Hz, 1H), 4.07-3.97 (m, 2H), 3.80-3.63 (m, 1H), 3.54-3.31 (m, 1H), 2.89-2.35 (m, 1H), 2.03 (hept, J=6.9 Hz, 1H), 1.88-1.69 (m, 1H), 1.69-1.52 (m, 3H), 1.52-1.36 (m, 3H), 1.36-1.04 (m, 2H), 0.97-0.85 (m, 10H), 0.75 (d, J=6.8 Hz, 3H), 0.69-0.52 (m, 1H). ¹³C NMR; IR (cm⁻¹); and Mass Spectrometry data were consistent with the expected data for the obtained compound. Olfactive description: Cheesy, glue.

### Compound 15: (7S,1OR)-3,7-diisopropyl-10-methyl-2,4-dioxaspiro[5.5]undecane

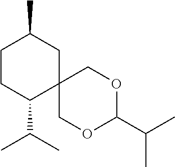 [SMILES: CC(C)C1OCC2(CO1)C[C@H](C)CC[C@H]2C(C)C]

To a 50 mL three-necked flask are added ((2S, 5R)-2-isopropyl-5-methylcyclohexane-1,1-diyl) dimethanol (10 g, 46.9 mmol), PTSA (0.446 g, 2.346 mmol), and isobutyraldehyde (13.53 g, 188 mmol). The medium is stirred at ambient temperature overnight and then transferred to a 250 mL three-necked flask equipped with a Dean-Stark trap. Cyclohexane (100 mL) is then added and the mixture is refluxed for 1 h. Water (50 mL) is added and the aqueous phase is extracted with MTBE (2×30 mL). The combined organic phases are washed with a saturated NaHCO₃ (40 mL) solution, brine (40 mL), dried over anhydrous MgSO₄, filtered, and concentrated. (7S, 1OR)-3,7-diisopropyl-10-methyl-2,4-dioxaspiro [5.5]undecane (3.4 g, 12.29 mmol, 26.2% yield) is recovered as a yellowish liquid after distillation (Teb=76° C., 0.8 torr; 2 GC peaks 65/35). ¹H NMR (300 MHz, CDCl₃) major isomer δ 4.18 (dd, J=15.0, 4.3 Hz, 1H), 4.04 (dd, J=11.1, 2.8 Hz, 1H), 3.99 (d, J=10.9 Hz, 1H), 3.85-3.66 (m, 1H), 3.49-3.30 (m, 1H), 2.61-2.48 (m, 1H), 2.11-1.93 (m, 1H), 1.89-1.65 (m, 2H), 1.65-1.43 (m, 2H), 1.40-1.00 (m, 3H), 1.00-0.92 (m, 6H), 0.92-0.89 (m, 3H), 0.89-0.86 (m, 3H), 0.78 (d, J=7.0 Hz, 3H), 0.65-0.42 (m, 1H). Minor isomer (characteristic signals) δ 4.35 (dd, J=12.0, 2.3 Hz, 1H), 2.93-2.70 (m, 1H). ¹³C NMR; IR (cm⁻¹); and Mass Spectrometry data were consistent with the expected data for the obtained compound. Olfactive description: Weak, chocolate.

### Compound 16: (7S,1OR)-3-(sec-butyl)-7-isopropyl-10-methyl-2,4-dioxaspiro[5.5]undecane

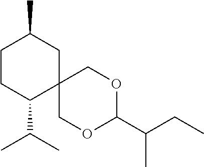 [SMILES: CCC(C)C1OCC2(CO1)C[C@H](C)CC[C@H]2C(C)C]

To a 50 mL three-necked flask of are added ((2S, 5R)-2-isopropyl-5-methylcyclohexane-1,1-diyl) dimethanol (10 g, 46.9 mmol), PTSA (0.446 g, 2.346 mmol), and 2-methylbutanal (16.17 g, 188 mmol). The medium is stirred at ambient temperature for 1 h, then sodium carbonate (0.497 g, 4.69 mmol) is added. After 5 min of stirring, the medium is filtered and distilled. (7S, 1OR)-3-(sec-butyl)-7-isopropyl-10-methyl-2,4-dioxaspiro [5.5]undecane (5.8 g, 20.53 mmol, 44% yield) is recovered as a colorless liquid (T=80° C., 0.4 torr, 2 GC peaks 15/85). ¹H NMR (300 MHz, CDCl₃) major isomer δ 4.39-4.19 (m, 1H), 4.09-3.93 (m, 2H), 3.80-3.64 (m, 1H), 3.49-3.29 (m, 1H), 2.63-2.44 (m, 1H), 2.12-1.93 (m, 1H), 1.84-1.68 (m, 1H), 1.67-1.40 (m, 3H), 1.38-1.04 (m, 3H), 0.97-0.94 (m, 1H), 0.95-0.86 (m, 12H), 0.87-0.83 (m, 1H), 0.83-0.71 (m, 3H), 0.68-0.47 (m, 1H). ¹³C NMR; IR (cm⁻¹); and Mass Spectrometry data were consistent with the expected data for the obtained compound. Olfactive description: Weak, ethereal.

### Compound 17: (2S,5R)-2-isopropyl-1,1-bis(methoxymethyl)-5-methylcyclohexane

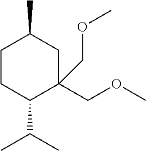 [SMILES: COCC1(COC)C[C@H](C)CC[C@H]1C(C)C]

In a nitrogen flushed 250 mL round bottomed flask equipped with a bubbler are added sodium hydride (3,51 g, 88 mmol), THF (165 ml) and then a solution of ((2S,5R)-2-isopropyl-5-methylcyclohexane-1,1-diyl)dimethanol (8 g, 39.9 mmol) in THF (35 ml) is added dropwise at room temperature over 15 min. Methyl iodide (6.24 ml, 100 mmol) is then added and the mixture refluxed overnight. Water (30 mL) is added and aqueous phase was extracted with MTBE (2×50 mL). Organic phases are gathered and washed with brine (30 mL), dried over anhydrous MgSO₄, filtered and concentrated. (2S,5R)-2-isopropyl-1,1-bis(methoxymethyl)-5-methylcyclohexane (6.4 g, 25.8 mmol, 64.6% yield) is obtained as a colorless liquid after distillation (T=60° C., 0.2 mbar). ¹H NMR (300 MHz, Chloroform-d) δ 3.44 (d, J=9.2 Hz, 1H), 3.39 (d, J=9.3 Hz, 1H), 3.33 (s, 3H), 3.30 (d, J=2.5 Hz, 3H), 3.28-3.20 (m, 2H), 2.03 (hept, J=6.9 Hz, 1H), 1.78-1.60 (m, 2H), 1.60-1.03 (m, 5H), 0.99 (d, J=12.8 Hz, 1H), 0.90 (d, J=6.9 Hz, 3H), 0.86 (d, J=6.5 Hz, 3H), 0.74 (d, J=6.8 Hz, 3H). ¹³C NMR; IR (cm⁻¹); and Mass Spectrometry data were consistent with the expected data for the obtained compound. Olfactive description: Fresh, weak, grapefruit.

### Compound 18: (7S,1OR)-7-isopropyl-10-methyl-3-phenyl-2,4-dioxaspiro[5.5]undecane

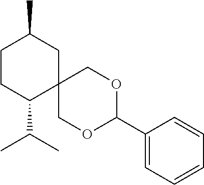 [SMILES: CC(C)[C@@H]1CC[C@@H](C)CC12COC(c1ccccc1)OC2]

To a 50 mL three-necked flask of are added ((2S, 5R)-2-isopropyl-5-methylcyclohexane-1,1-diyl) dimethanol (10 g, 46.9 mmol), PTSA (0.451 g, 2.371 mmol), and benzaldehyde (5.54 g, 52.2 mmol). The medium is stirred at ambient temperature for 1 h, then sodium carbonate (0.503 g, 4.74 mmol) is added. After 5 min of stirring, the medium is filtered and distilled. (7S,1OR)-7-isopropyl-10-methyl-3-phenyl-2,4-dioxaspiro[5.5]undecane (7.8 g, 25.4 mmol, 53.6% yield) is obtained as a colorless viscous oil (T=120° C., 0.6 torr, 2 GC peaks 26/74). ¹H NMR (300 MHz, CDCl3) δ 7.61-7.46 (m, 2H), 7.45-7.29 (m, 3H), 5.44 (d, J=8.9 Hz, 1H), 4.29-4.17 (m, 1H), 4.05-3.88 (m, 1H), 3.85-3.41 (m, 2H), 2.86-2.67 (m, 1H), 2.23-2.03 (m, 1H), 1.89-1.74 (m, 1H), 1.74-1.47 (m, 2H), 1.31-1.19 (m, 1H), 1.10-0.93 (m, 7H), 0.92-0.79 (m, 4H), 0.79-0.60 (m, 1H). ¹³C NMR; IR (cm⁻¹); and Mass Spectrometry data were consistent with the expected data for the obtained compound. Olfactive description: Weak, almond.

### Compound 19: (7S,1OR)-3,3-diethyl-7-isopropyl-10-methyl-2,4-dioxaspiro[5.5]undecane

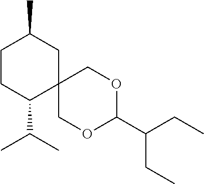 [SMILES: CCC(CC)C1OCC2(CO1)C[C@H](C)CC[C@H]2C(C)C]

In a 250 mL round bottomed flask equipped with a Dean-Stark apparatus are introduced ((2S,5R)-2-isopropyl-5-methylcyclohexane-1,1-diyl)dimethanol (10 g, 47.4 mmol), PTSA (0.451 g, 2.371 mmol), pentan-3-one (16.34 g, 190 mmol) and cyclohexane (100 mL). The mixture is refluxed 1 h and then cooled, washed with sat. NaHCO₃ (40 mL), brine (40 mL), dried over anhydrous MgSO₄, filtered and distilled (T=100° C., 0.7 torr). (7S,1OR)-3,3-diethyl-7-isopropyl-10-methyl-2,4-dioxaspiro[5.5]undecane (5.8 g, 20.31 mmol, 42.8% yield) is obtained as a colorless liquid. ¹H NMR (300 MHz, CDCl₃) δ 3.99-3.82 (m, 2H), 3.64 (dd, 1H), 3.19 (dd, 1H), 2.35-2.16 (m, 2H), 1.81-1.60 (m, 5H), 1.54-1.37 (m, 2H), 1.20-1.05 (m, 1H), 0.94-0.79 (m, 17H), 0.61-0.46 (m, 1H). ¹³C NMR; IR (cm⁻¹); and Mass Spectrometry data were consistent with the expected data for the obtained compound. Olfactive description: Weak, fresh, woody.

### Compound 20: (3,3-dimethylcyclohexane-1,1-diyl)dimethanol

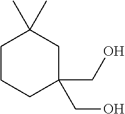

In a 100 ml three-necked flask are added potassium hydroxide (0.600 g, 10.70 mmol) and ethanol (75 ml). After complete solubilization of the KOH, 37% aqueous formaldehyde (0.796 ml, 10.70 mmol) is added dropwise at ambient temperature, and then the mixture is stirred for 10 min. 3,3-dimethylcyclohexane-1-carbaldehyde (1 g, 7.13 mmol) is then added dropwise over 30 min and the mixture is stirred 72 h at ambient temperature. The medium is concentrated and the aqueous phase extracted with MTBE (2×30 mL). The combined organic phases are washed sequentially with a saturated NaHCO₃ (30 mL) solution, and then brine (30 mL), dried over anhydrous MgSO₄, filtered, and concentrated. (3,3-dimethylcyclohexane-1,1-diyl)dimethanol (0.64 g, 7.13 mmol, 52% yield) is recovered in the form of a white solid after purification by chromatographic column (CombiFlash®, cyclohexane/EtOAc gradient). ¹H NMR (300 MHz, Chloroform-d) δ 3.67-3.51 (m, 4H), 3.06 (s, 2H), 1.56-1.42 (m, 2H), 1.31-1.17 (m, 6H), 0.94 (s, 6H). ¹³C NMR; IR (cm⁻¹); and Mass Spectrometry data were consistent with the expected data for the obtained compound. Olfactive description: Almost odorless, solvent.

### Compound 21: 4,4′-((((2S,5R)-2-isopropyl-5-methylcyclohexane-1,1-diyl)bis (methylene))bis(oxy))bis(4-oxobutanoic acid)

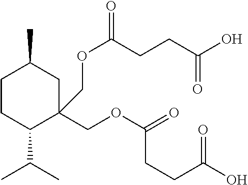 [SMILES: CC(C)[C@@H]1CC[C@@H](C)CC1(COC(=O)CCC(=O)O)COC(=O)CCC(=O)O]

In a 50 ml three-necked flask are added ((2S,5R)-2-isopropyl-5-methylcyclohexane-1,1-diyl)dimethanol (5 g, 24.96 mmol), MCH (10 ml) then succinic anhydride (5.00 g, 49.9 mmol). The mixture is refluxed overnight and then concentrated. 4,4′-((((2R,5R)-2-isopropyl-5-methylcyclohexane-1,1-diyl)bis(methylene))bis(oxy))bis(4-oxobutanoic acid) (7.25 g, 17.20 mmol, 68.9% yield) is obtained as a viscous oil after purification by chromatographic column (CombiFlash®, cyclohexane/EtOAc gradient). ¹H NMR (300 MHz, Chloroform-d) δ 10.97 (s, 2H), 4.31 (d, J=11.4 Hz, 1H), 4.18-3.98 (m, 4H), 2.74-2.53 (m, 8H), 1.99 (q, J=6.9 Hz, 1H), 1.84-1.60 (m, 2H), 1.60-1.40 (m, 2H), 1.40-1.30 (m, 2H), 0.89 (d, J=6.8 Hz, 4H), 0.84 (d, J=6.4 Hz, 3H), 0.75 (d, J=6.8 Hz, 3H). ¹³C NMR and IR (cm⁻¹) data were consistent with the expected data for the obtained compound. Olfactive description: Odorless.

### Compound 22: (7S,1OR)-7-isopropyl-3,10-dimethyl-3-phenyl-2,4-dioxaspiro[5.5]undecane

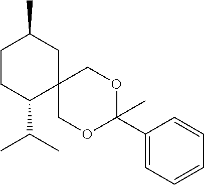 [SMILES: CC(C)[C@@H]1CC[C@@H](C)CC12COC(C)(c1ccccc1)OC2]

To a 50 mL three-necked flask are ((2S,5R)-2-isopropyl-5-methylcyclohexane-1,1-diyl)dimethanol (10 g, 47.4 mmol), PTSA (0.451 g, 2.371 mmol), and acetophenone (6.27 g, 52.2 mmol). The medium is stirred at ambient temperature overnight and then transferred to a 250 mL three-necked flask equipped with a Dean-Stark trap. Cyclohexane (100 mL) is then added and the mixture is stirred overnight at room temperature and then refluxed 16 h. The cooled solution was washed with a saturated NaHCO₃ (40 mL) solution, brine (40 mL), dried over anhydrous MgSO₄, filtered, and concentrated. (7S,1OR)-7-isopropyl-3,10-dimethyl-3-phenyl-2,4-dioxaspiro[5.5]undecane (3.6 g, 11.21 mmol, 23.6% yield) is recovered as a viscous colorless liquid after distillation (Teb=97° C., 0.6 torr; 2 GC peaks 61/39). ¹H NMR (300 MHz, CDCl₃) major isomer δ 7.25-7.13 (m, 4H), 7.13-7.00 (m, 1H), 3.93-3.62 (m, 1H), 3.60-3.50 (m, 1H), 3.51-3.03 (m, 1H), 3.03-2.62 (m, 1H), 2.62-2.29 (m, 1H), 1.63-1.34 (m, 1H), 1.29 (d, J=9.7 Hz, 3H), 1.24-0.83 (m, 2H), 0.78 (d, J=6.9 Hz, 3H), 0.73 (d, J=6.4 Hz, 3H), 0.69-0.55 (m, 2H), 0.54-0.48 (m, 3H), 0.46-0.35 (m, 1H), 0.30 (d, J=6.8 Hz, 2H), 0.20 (t, J=12.9 Hz, OH). ¹³C NMR; IR (cm⁻¹); and Mass Spectrometry data were consistent with the expected data for the obtained compound. Olfactive description: Powdery, vanilla, mimosa.

### Compound 23: 2-((7S,10R)-7-isopropyl-10-methyl-2,4-dioxaspiro[5.5]undecan-3-yl) pyridine

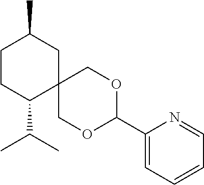 [SMILES: CC(C)[C@@H]1CC[C@@H](C)CC12COC(c1ccccn1)OC2]

To a 50 mL three-necked flask are ((2S,5R)-2-isopropyl-5-methylcyclohexane-1,1-diyl)dimethanol (10 g, 47.4 mmol), PTSA (0.451 g, 2.371 mmol), and picolinaldehyde (5.59 g, 52.2 mmol). The medium is stirred at ambient temperature overnight and then transferred to a 250 mL three-necked flask equipped with a Dean-Stark trap. Cyclohexane (100 mL) is then added and the mixture is refluxed overnight. The cooled solution was washed with a saturated NaHCO₃ (40 mL) solution, brine (40 mL), dried over anhydrous MgSO₄, filtered, and concentrated. 2-((7S,10R)-7-isopropyl-10-methyl-2,4-dioxaspiro[5.5]undecan-3-yl)pyridine (4.8 g, 15.76 mmol, 33.2% yield) is recovered as a viscous yellow liquid after distillation (Teb=110° C., 0.4 torr; 2 GC peaks 72/28). ¹H NMR (300 MHz, CDCl₃) major isomer δ 8.38 (tdd, J=5.9, 2.4, 1.0 Hz, 1H), 7.52 (tt, J=7.7, 1.6 Hz, 1H), 7.46-7.30 (m, 1H), 7.10-6.99 (m, 1H), 5.28 (d, J=2.2 Hz, 1H), 4.27-3.82 (m, 2H), 3.83-3.63 (m, 1H), 3.36 (dd, J=10.9, 2.7 Hz, 1H), 2.49 (ddd, J=13.6, 3.4, 2.4 Hz, 1H), 1.62-1.48 (m, 1H), 1.40 (dtd, J=12.0, 5.7, 2.7 Hz, 1H), 1.30 (dq, J=13.4, 3.5 Hz, 1H), 1.03-0.94 (m, 1H), 0.94-0.76 (m, 1H), 0.72 (td, J=6.9, 5.8 Hz, 7H), 0.61 (dd, J=6.6, 4.2 Hz, 4H), 0.47 (ddd, J=14.4, 12.5, 2.1 Hz, 1H). ¹³C NMR; IR (cm⁻¹); and Mass Spectrometry data were consistent with the expected data for the obtained compound. Olfactive description: Green, mimosa.

### Compound 24: (7S,1OR)-7-isopropyl-10-methyl-3-(thiophen-2-yl)-2,4-dioxaspiro[5.5]undecane

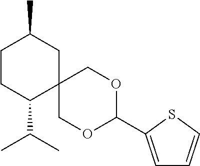 [SMILES: CC(C)[C@@H]1CC[C@@H](C)CC12COC(c1cccs1)OC2]

To a 50 mL three-necked flask are ((2S,5R)-2-isopropyl-5-methylcyclohexane-1,1-diyl)dimethanol (10 g, 47.4 mmol), PTSA (0.451 g, 2.371 mmol), and thiophene-2-carbaldehyde (5.85 g, 52.2 mmol). The medium is stirred at ambient temperature 27 h. Sodium carbonate (0.503 g, 4.74 mmol) is added and then water (15 mL). The organic layer is washed with brine (40 mL), dried over anhydrous MgSO₄, filtered, and concentrated. (7S,1OR)-7-isopropyl-10-methyl-3-(thiophen-2-yl)-2,4-dioxaspiro[5.5]undecane (3.9 g, 12.37 mmol, 26.1% yield) is recovered as a yellow liquid after distillation (Teb=70° C., 0.9 torr; 2 GC peaks 35/65). ¹H NMR (300 MHz, CDCl3) major isomer δ 7.32 (dd, J=5.0, 1.3 Hz, 1H), 7.21-7.10 (m, 1H), 7.01 (dt, J=4.8, 3.8 Hz, 1H), 5.70 (s, 1H), 4.58-4.13 (m, 2H), 4.03-3.84 (m, 1H), 3.67-3.51 (m, 1H), 2.85-2.67 (m, 1H), 2.10 (hept, J=6.8 Hz, 1H), 1.82 (dp, J=12.8, 3.4 Hz, 1H), 1.70-1.60 (m, 1H), 1.60-1.50 (m, 1H), 1.23 (ddd, J=13.2, 4.6, 2.5 Hz, 1H), 1.06-0.92 (m, 7H), 0.86 (t, J=6.9 Hz, 4H), 0.74-0.59 (m, 1H). ¹³C NMR; IR (cm⁻¹); and Mass Spectrometry data were consistent with the expected data for the obtained compound. Olfactive description: Weak, green, bread crust.

### Compound 25: (7S,1OR)-7-isopropyl-10-methyl-3-(2-(methylthio)ethyl)-2,4-dioxaspiro [5.5]undecane

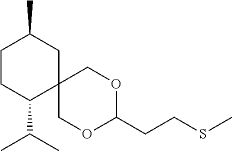 [SMILES: CSCCC1OCC2(CO1)C[C@H](C)CC[C@H]2C(C)C]

To a 50 mL three-necked flask are ((2S,5R)-2-isopropyl-5-methylcyclohexane-1,1-diyl)dimethanol (10 g, 47.4 mmol), PTSA (0.451 g, 2.371 mmol), 3-(methylthio)propanal (9.88 g, 95 mmol) and cyclohexane (7 mL). The medium is stirred at ambient temperature 27 h. The mixture was washed with a saturated NaHCO₃ (40 mL) solution, brine (40 mL), dried over anhydrous MgSO₄, filtered, and concentrated. (7S,1OR)-7-isopropyl-10-methyl-3-(2-(methylthio)ethyl)-2,4-dioxaspiro[5.5]undecane (0.5 g, 1.653 mmol, 3.49% yield) is recovered as a colorless liquid after distillation (Teb=77° C., 0.8 torr) and chromatographic column (CombiFlash®, cyclohexane/EtOAc gradient, 2 GC peaks 41/59). ¹H NMR (300 MHz, CDCl₃) major isomer δ 4.61-4.51 (m, 1H), 4.37-3.93 (m, 2H), 3.77-3.63 (m, 1H), 3.50-3.31 (m, 1H), 2.86-2.40 (m, 3H), 2.10 (s, 3H), 2.01 (p, J=6.9 Hz, 1H), 1.97-1.81 (m, 2H), 1.75 (dh, J=12.6, 3.1 Hz, 1H), 1.65-1.39 (m, 1H), 1.39-1.18 (m, 1H), 1.18-0.93 (m, 3H), 0.94-0.66 (m, 9H), 0.66-0.43 (m, 1H). ¹³C NMR; IR (cm⁻¹); and Mass Spectrometry data were consistent with the expected data for the obtained compound. Olfactive description: Metallic, garlic, sardine.

### Compound 26: 3,8,8-trimethyl-2,4-dioxaspiro[5.5]undecane

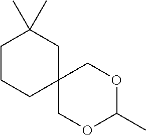

To a 50 mL three-necked flask are (3,3-dimethylcyclohexane-1,1-diyl)dimethanol (8 g, 46.4 mmol), acetaldehyde (6.14 g, 139 mmol) and PTSA (0.442 g, 2.322 mmol). The mixture is stirred at ambient temperature 17 h. Sodium bicarbonate (0.5 g) is added and the mixture is filtered then concentrated. 3,8,8-trimethyl-2,4-dioxaspiro[5.5]undecane (9.21 g, 23.72 mmol, 51.1% yield) is recovered as a colorless liquid after distillation (Teb=57° C., 1.2 torr; 2 GC peaks 53/47). ¹H NMR (300 MHz, CDCl₃) major isomer δ 4.58 (dq, J=6.6, 5.0 Hz, 1H), 3.95-3.76 (m, 2H), 3.42-3.23 (m, 2H), 1.73-1.64 (m, 1H), 1.62 (s, 1H), 1.56-1.47 (m, 1H), 1.46-1.38 (m, 1H), 1.37-1.14 (m, 6H), 1.00-0.97 (m, 3H), 0.87 (s, 4H). ¹³C NMR; IR (cm⁻¹); and Mass Spectrometry data were consistent with the expected data for the obtained compound. Olfactive description: Camphor, violet, frais.

### Compound 27: (2-(sec-butyl)cyclohexane-1,1-diyl)dimethanol

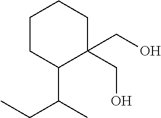

In a 100 ml three-necked flask are added potassium hydroxide (17 g, 10.70 mmol) and ethanol (200 ml). After almost complete solubilization of the KOH, 37% aqueous formaldehyde (19.47 ml, 261 mmol) is added dropwise at ambient temperature, and then the mixture is stirred for 10 min. 2-(sec-butyl)cyclohexane-1-carbaldehyde (11 g, 65.4 mmol) is then added dropwise over 30 min and the mixture is stirred 72 h at ambient temperature. The medium is concentrated and the aqueous phase extracted with MTBE (2×100 mL). The combined organic phases are washed sequentially with a saturated NaHCO₃ (150 mL) solution, and then brine (100 mL), dried over anhydrous MgSO₄, filtered, and concentrated. (2-(sec-butyl)cyclohexane-1,1-diyl)dimethanol (0.9 g, 4.27 mmol, 6.53% yield) is recovered in the form of a colorless liquid after distillation (Teb=119° C., 1 torr) followed by column chromatography (CombiFlash®, cyclohexane/EtOAc gradient). ¹H NMR (300 MHz, Chloroform-d) δ 4.18-4.03 (m, 1H), 3.98-3.74 (m, 2H), 3.70-3.44 (m, 1H), 3.44-3.27 (m, 1H), 2.32-2.15 (m, 1H), 1.95-1.70 (m, 2H), 1.69-1.49 (m, 2H), 1.49-1.32 (m, 1H), 1.32-1.06 (m, 5H), 1.06-0.90 (m, 1H), 0.90-0.77 (m, 5H), 0.77-0.66 (m, 2H). ¹³C NMR; IR (cm⁻¹); and Mass Spectrometry data were consistent with the expected data for the obtained compound. Olfactive description: Almost odorless, solvent.

### Compound 28: Ethyl (7S,1OR)-7-isopropyl-10-methyl-2,4-dioxaspiro[5.5]undecane-3-carboxylate

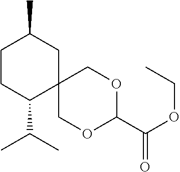 [SMILES: CCOC(=O)C1OCC2(CO1)C[C@H](C)CC[C@H]2C(C)C]

To a 100 mL three-necked flask equipped with a Dean-Stark trap are ((5R)-2-isopropyl-5-methylcyclohexane-1,1-diyl)dimethanol (5 g, 24.96 mmol), ethyl 2-glyoxalate 50% in toluene (10.19 ml, 49.9 mmol) puis PTSA (0.237 g, 1.248 mmol). The medium is stirred at ambient temperature 3 h30. Toluene (300 mL) is then added and the mixture is refluxed 1 h. To the cooled solution is added sodium carbonate (0.265 g, 2.496 mmol) and stirred for 5 min. The solution is filtered, and concentrated. ethyl (1OR)-7-isopropyl-10-methyl-2,4-dioxaspiro[5.5]undecane-3-carboxylate (0.3 g, 0.992 mmol, 4% yield) is recovered as a colorless liquid (2 GC peaks 48/52) after distillation (Teb=100° C., 0.3 mbar) followed by column chromatography (CombiFlash®, cyclohexane/EtOAc gradient). ¹H NMR (300 MHz, CDCl₃) δ 4.97 (s, OH), 4.92 (s, 1H), 4.42-3.80 (m, 5H), 3.62-3.44 (m, 1H), 2.68-2.45 (m, 1H), 2.00 (hept, J=7.0 Hz, 1H), 1.76 (dq, J=12.5, 2.9 Hz, 1H), 1.63-1.42 (m, 2H), 1.33 (td, J=7.1, 3.5 Hz, 3H), 1.27-1.02 (m, 2H), 0.95-0.87 (m, 5H), 0.84 (dd, J=6.5, 2.8 Hz, 3H), 0.78 (d, J=6.8 Hz, 2H), 0.70-0.54 (m, 1H). ¹³C NMR; IR (cm⁻¹); and Mass Spectrometry data were consistent with the expected data for the obtained compound. Olfactive description: Odorless.

### Compound 29: 2,2′-((2S,5R)-2-isopropyl-5-methylcyclohexane-1,1-diyl)bis(ethan-1-ol)

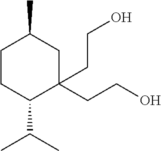 [SMILES: CC(C)[C@@H]1CC[C@@H](C)CC1(CCO)CCO]

To a 250 mL three-necked flask at 0° C. under nitrogen are added (methoxymethyl)triphenylphosphonium chloride (20.96 g, 61.1 mmol), THF (100 ml) and Potassium t-butoxide (6.86 g, 61.1 mmol). The mixture turned red upon stirring 30 min at room temperature. A solution of (2S,5R)-2-isopropyl-5-methylcyclohexane-1,1-dicarbaldehyde (5 g, 25.5 mmol) in THF (20 ml) is then added over 20 min at 0° C. and stirred overnight at room temperature. MTBE (100 mL) and 10% aqueous HCl (150 mL) are added. Aqueous phase is extracted with MTBE (200 mL). The organic phases are gathered, washed with brine (30 mL), dried over anhydrous MgSO₄, filtered and concentrated. Cyclohexane (100 mL) is added and the white precipitate is filtered off. The filtrate is concentrated and distilled (0.3 mbar, T=66-80° C.) to yield (2S,5R)-2-isopropyl-1,1-bis(2-methoxyvinyl)-5-methylcyclohexane (2.2 g, 4.79 mmol, 18.8% yield) as a yellowish liquid as a mixture of isomers accounting for 55% GC. This product is diluted in THF (40 ml) and 10% aqueous HCl (20 mL) is added. The mixture is stirred 2 h at room temperature then poured into MTBE 100 mL). The organic phase is washed with saturated NaHCO₃ (50 mL), brine (20 mL), dried on anhydrous MgSO₄, filtered and concentrated. 2,2′-((2S,5R)-2-isopropyl-5-methylcyclohexane-1,1-diyl)diacetaldehyde (0.3 g, 1.244 mmol, 4.9% yield) is recovered as a yellow oil after purification by column chromatography (CombiFlash®, cyclohexane/EtOAc gradient). The product is diluted in EtOH (20 mL) and sodium borohydride (0.482 g, 12.74 mmol) is added. The mixture is stirred overnight and concentrated. MTBE (30 mL) and 10% aqueous NaOH (20 mL) are added. The organic phase is washed with saturated brine (20 mL), dried on anhydrous MgSO₄, filtered and concentrated. 2,2′-((2S,5R)-2-isopropyl-5-methylcyclohexane-1,1-diyl)bis(ethan-1-ol) (0.25 g, 1.084 mmol, 4.25% yield) is recovered as a white solid after purification by column chromatography (CombiFlash®, cyclohexane/EtOAc gradient). ¹H NMR (300 MHz, CDCl₃) δ 3.77-3.58 (m, 4H), 2.15 (s, 2H), 2.06-1.89 (m, 2H), 1.89-1.79 (m, 1H), 1.79-1.69 (m, 1H), 1.69-1.54 (m, 1H), 1.54-1.45 (m, 1H), 1.45-1.40 (m, 2H), 1.38 (d, J=3.3 Hz, 1H), 1.35-1.31 (m, 1H), 1.30-1.22 (m, 1H), 1.18-1.09 (m, 1H), 1.06-0.94 (m, 1H), 0.90 (d, J=6.9 Hz, 3H), 0.83 (d, J=1.9 Hz, 3H), 0.81 (d, J=2.2 Hz, 3H), 0.74 (dd, J=12.3, 4.7 Hz, OH). ³C NMR; IR (cm⁻¹); and Mass Spectrometry data were consistent with the expected data for the obtained compound. Olfactive description: Almost odorless, solvent.

### Compound 30: (2-isopropyl-5,5-dimethylcyclohexane-1,1-diyl)dimethanol

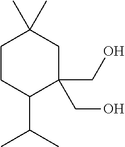

In a 100 ml three-necked flask are added potassium hydroxide (7.39 g, 132 mmol) and ethanol (55 ml). After almost complete solubilization of the KOH, 37% aqueous formaldehyde (9.8 ml, 132 mmol) is added dropwise at ambient temperature, and then the mixture is stirred for 10 min. 2-isopropyl-5,5-dimethylcyclohexane-1-carbaldehyde (6 g, 32.9 mmol) (synthetized according to WO2016153011) is then added dropwise over 30 min and the mixture is stirred 24 h at ambient temperature. 37% aqueous Formaldehyde (9.80 ml, 132 mmol) and potassium hydroxide (7.39 g, 132 mmol) are added one more time and the mixture is stirred at room temperature overtime. The medium is concentrated and the aqueous phase extracted with MTBE (2×30 mL). The combined organic phases are washed sequentially with a saturated NaHCO₃ (30 mL) solution, and then brine (30 mL), dried over anhydrous MgSO₄, filtered, and concentrated. ((2-isopropyl-5,5-dimethylcyclohexane-1,1-diyl)dimethanol (3 g, 11.20 mmol, 34,0% yield) is recovered in the form of a white solid after purification by distillation (P=0.3 mbar, T=120° C.) and chromatographic column (CombiFlash®, cyclohexane/EtOAc gradient) (mixture of diastereoisomers 5/95). ¹H NMR (300 MHz, Chloroform-d) δ 4.06-3.74 (m, 3H), 3.31 (dd, J=10.3, 1.8 Hz, 1H), 2.79 (s, 2H), 2.04 (dd, J=14.1, 2.5 Hz, 1H), 1.99-1.85 (m, 1H), 1.56-1.45 (m, 1H), 1.45-1.30 (m, 2H), 1.22-1.04 (m, 3H), 1.02 (s, 3H), 0.93 (s, 3H), 0.89 (d, J=6.9 Hz, 3H), 0.77 (d, J=6.8 Hz, 3H). ¹³C NMR; IR (cm⁻¹); and Mass Spectrometry data were consistent with the expected data for the obtained compound. Olfactive description: Odorless.

### Compound 31: 7-(sec-butyl)-3-methyl-2,4-dioxaspiro[5.5]undecane

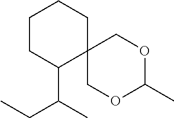 [SMILES: CCC(C)C1CCCCC12COC(C)OC2]

To a 50 mL three-necked flask are (2-(sec-butyl)cyclohexane-1,1-diyl)dimethanol (3.6 g, 15.28 mmol), acetaldehyde (2.56 ml, 45.8 mmol) et p-toluenesulfonic acid monohydrate (0.145 g, 0.764 mmol). The mixture is stirred at ambient temperature overnight. Sodium bicarbonate (0.5 g) is added and the mixture is filtered then concentrated. 7-(sec-butyl)-3-methyl-2,4-dioxaspiro[5.5]undecane (0.6 g, 2.60 mmol, 17% yield) is recovered as a colorless liquid (mixture of isomers) after distillation (Teb=64° C., 1 torr) and column chromatography (CombiFlash®, cyclohexane/EtOAc gradient). ¹H NMR (300 MHz, CDCl₃) δ 4.65-4.51 (m, 1H), 4.34 (ddd, J=15.1, 12.0, 2.5 Hz, OH), 4.15-3.93 (m, 2H), 3.82-3.59 (m, 1H), 3.44-3.32 (m, 1H), 2.64-2.50 (m, 1H), 1.84-1.35 (m, 6H), 1.31 (t, J=5.3 Hz, 3H), 1.28-0.95 (m, 5H), 0.95-0.87 (m, 3H), 0.87-0.80 (m, 2H), 0.76 (d, J=6.9 Hz, 1H). ³C NMR; IR (cm⁻¹); and Mass Spectrometry data were consistent with the expected data for the obtained compound. Olfactive description: Animalic, blackcurrent, ambery.

### Compound 32: ((2S,5R)-1-(hydroxymethyl)-2-isopropyl-5-methylcyclohexyl)methyl acetate

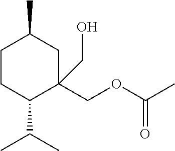 [SMILES: CC(=O)OCC1(CO)C[C@H](C)CC[C@H]1C(C)C]

To a 100 mL three-necked flask at 0° C. are added ((2S,5R)-2-isopropyl-5-methylcyclohexane-1,1-diyl)dimethanol (2 g, 9.98 mmol), MTBE (50 ml) and triethylamine (2.087 ml, 14.98 mmol). Acetyl chloride (0.784 g, 9.98 mmol) is added dropwise over 5 min and the mixture is stirred at room temperature for 30 min. Water (20 mL) is added and the organic layer is washed with brine, dried over anhydrous MgSO₄, filtered and concentrated. ((2S,5R)-1-(hydroxymethyl)-2-isopropyl-5-methylcyclohexyl)methyl acetate (0.72 g, 2.94 mmol, 29.5% yield) (mixture of diastereoisomers 59/41) is recovered as a colorless sticky oil. ¹H NMR (300 MHz, CDCl₃) δ 4.32-4.02 (m, 2H), 3.79-3.41 (m, 2H), 2.09 (d, J=7.0 Hz, 3H), 2.23-1.91 (m, 1H), 2.01 (s, 1H), 1.93-1.60 (m, 2H), 1.60-1.40 (m, 2H), 1.40-1.17 (m, 2H), 0.96-0.82 (m, 7H), 0.80 (q, J=3.9 Hz, 1H), 0.75 (dd, J=6.8, 3.7 Hz, 3H). ¹³C NMR; IR (cm⁻¹); and Mass Spectrometry data were consistent with the expected data for the obtained compound. Olfactive description: Almost odorless, fresh.

### Compound 33: 7-isopropyl-3,10,10-trimethyl-2,4-dioxaspiro[5.5]undecane

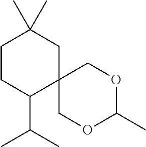 [SMILES: CC1OCC2(CO1)CC(C)(C)CCC2C(C)C]

To a 50 mL three-necked flask are added (2-isopropyl-5,5-dimethylcyclohexane-1,1-diyl)dimethanol (1 g, 4.67 mmol), acetaldehyde (1.565 ml, 28.0 mmol) and p-toluenesulfonic acid monohydrate (0.044 g, 0.233 mmol). The mixture is stirred at ambient temperature overnight. Sodium carbonate (0.049 g, 0.467 mmol) is added and the mixture is filtered then concentrated. 7-isopropyl-3,10,10-trimethyl-2,4-dioxaspiro[5.5]undecane (0.3 g, 1.211 mmol, 25.9% yield) is recovered as a colorless liquid after column chromatography (CombiFlash®, cyclohexane/EtOAc gradient). ¹H NMR (300 MHz, CDCl₃) δ 4.61 (q, J=5.0 Hz, 1H), 4.18 (dd, J=11.3, 3.1 Hz, 1H), 3.90 (d, J=10.8 Hz, 1H), 3.77-3.55 (m, 1H), 3.31 (dd, J=10.8, 3.1 Hz, 1H), 2.43 (dd, J=14.3, 2.5 Hz, 1H), 2.01 (hept, J=6.9 Hz, 1H), 1.48 (dq, J=12.5, 3.1 Hz, 1H), 1.43-1.36 (m, 1H), 1.34 (d, J=5.1 Hz, 3H), 1.31-1.22 (m, 1H), 1.19-1.06 (m, 1H), 1.03 (s, 3H), 0.94 (s, 3H), 0.92 (d, J=7.0 Hz, 4H), 0.88-0.82 (m, 1H), 0.80 (d, J=6.8 Hz, 3H). ¹³C NMR; IR (cm⁻¹); and Mass Spectrometry data were consistent with the expected data for the obtained compound. Olfactive description: Woody, dirty, ambery.

### Compound 34: 1,1′-((2S,5R)-2-isopropyl-5-methylcyclohexane-1,1-diyl)bis(ethan-1-ol)

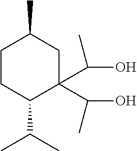 [SMILES: CC(C)[C@@H]1CC[C@@H](C)CC1(C(C)O)C(C)O]

To a 500 mL three-necked flask under nitrogen are added THF (200 ml) and methylmagnesium chloride 3M in THF (37.8 ml, 113 mmol). A solution of (2S,5R)-2-isopropyl-5-methylcyclohexane-1,1-dicarbaldehyde (10 g, 45.3 mmol) in THF (100 ml) is then added dropwise at 0° C. over 30 min. The mixture is stirred 30 min and poured into a mixture of sat. aqueous NH₄Cl (200 mL) and ice. The organic layer is washed with brine, dried over anhydrous MgSO₄, filtered and concentrated. 1,1′-((2S,5R)-2-isopropyl-5-methylcyclohexane-1,1-diyl)bis(ethan-1-ol) (0.3 g, 1.314 mmol, 2.90% yield) is obtained as a white solid after distillation (P=0.5 mbar, T=104° C.) and recrystallization in cyclohexane (5 mL). ¹H NMR (300 MHz, CDCl₃) δ 4.58 (dd, 1H), 4.24 (dd, 1H), 2.24 (s, 2H), 2.16-2.00 (m, 1H), 1.80-1.64 (m, 2H), 1.59-1.46 (m, 1H), 1.40 (d, 3H), 1.39-1.27 (m, 2H), 1.15 (d, J=6.5 Hz, 3H), 1.12-1.04 (m, 1H), 0.95-0.83 (m, 9H), 0.82-0.66 (m, 2H). ¹³C NMR; IR (cm⁻¹); and Mass Spectrometry data were consistent with the expected data for the obtained compound. Olfactive description: Almost odorless, fresh.

### Compound 35: 2-((1R,2R,5R)-5-methyl-2-(prop-1-en-2-yl)cyclohexyl)propane-1,3-diol

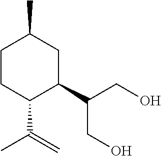

To a 250 mL three-necked flask under nitrogen are added dimethyl 2-((1R,2R,5R)-5-methyl-2-(prop-1-en-2-yl)cyclohexyl)malonate (10 g, 33.5 mmol) (Organic Syntheses, Coll. Vol. 9, p.310 (1998); Vol. 71, p.167 (1993)) and MTBE (130 mL). Sodium bis(2-methoxyethoxy)aluminum hydride 70% in toluene (40.2 ml, 141 mmol) is added dropwise while keeping the temperature below 10° C. The mixture is stirred at ambient temperature overnight. The mixture is carefully poured into a mixture of 10% aqueous HCl (200 mL) and ice. The aqueous phase is extracted with MTBE (2×100 mL) and the combined organic phases are washed with saturated aqueous NaHCO₃ (100 mL), brine (100 mL), dried over anhydrous MgSO₄, filtered and concentrated. 2-((1R,2R,5R)-5-methyl-2-(prop-1-en-2-yl)cyclohexyl)propane-1,3-diol (4.4 g, 19.27 mmol, 57.5% yield) is recovered as a colorless liquid after recrystallization in refluxing cyclohexane (10 mL). ¹H NMR (300 MHz, CDCl₃) δ 4.76 (s, 2H), 3.95-3.71 (m, 2H), 3.70-3.53 (m, 2H), 2.90 (s, 2H), 2.02-1.82 (m, 2H), 1.74-1.66 (m, 1H), 1.65 (s, 3H), 1.62-1.22 (m, 5H), 0.99-0.90 (m, 1H), 0.87 (d, J=6.5 Hz, 3H), 0.72-0.53 (m, 1H). ¹³C NMR; IR (cm⁻¹); and Mass Spectrometry data were consistent with the expected data for the obtained compound. Olfactive description: Almost odorless, aromatic.

### Compound 36: 2-methyl-5-((1R,2R,5R)-5-methyl-2-(prop-1-en-2-yl)cyclohexyl)-1,3-dioxane

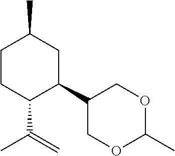 [SMILES: C=C(C)[C@@H]1CC[C@@H](C)C[C@H]1C1COC(C)OC1]

To a 50 mL three-necked flask are added 2-((1R,2R,5R)-5-methyl-2-(prop-1-en-2-yl)cyclohexyl)propane-1,3-diol (2 g, 9.42 mmol), acetaldehyde (1.245 g, 28.3 mmol) et p-toluenesulfonic acid monohydrate (0.090 g, 0.471 mmol). The mixture is stirred at ambient temperature overnight. Sodium bicarbonate (0.05 g) is added and the mixture is filtered then concentrated. 2-methyl-5-((1R,2R,5R)-5-methyl-2-(prop-1-en-2-yl)cyclohexyl)-1,3-dioxane (0.64 g, 2.66 mmol, 28.2% yield) is recovered as a colorless liquid after column chromatography (CombiFlash®, cyclohexane/EtOAc gradient). ¹H NMR (300 MHz, CDCl₃) δ 4.74 (s, 2H), 4.61-4.46 (m, 1H), 4.00 (dq, 1H), 3.80 (dq, 1H), 3.67 (t, J=11.3 Hz, 1H), 3.56 (t, J=11.1 Hz, 1H), 2.11-1.95 (m, 1H), 1.83 (td, J=11.6, 3.4 Hz, 1H), 1.71-1.52 (m, 6H), 1.41-1.20 (m, 6H), 0.97-0.78 (m, 4H), 0.68 (q, J=12.2 Hz, 1H). ¹³C NMR; IR (cm⁻¹); and Mass Spectrometry data were consistent with the expected data for the obtained compound. Olfactive description: Almost odorless, solvent.

### Compound 37: 2-((1R,2S,5R)-2-isopropyl-5-methylcyclohexyl)propane-1,3-diol

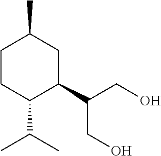

Step 1: In a 300 mL autoclave are added dimethyl 2-((1R,2R,5R)-5-methyl-2-(prop-1-en-2-yl)cyclohexyl)malonate (8 g, 29.8 mmol), Methanol (100 ml) and 10% Pd/C (1 g, 0.470 mmol). The mixture is stirred 24 h under H₂ (5 bar) at room temperature. The mixture is filtered through celite and then concentrated. Dimethyl 2-((1R,2S,5R)-2-isopropyl-5-methylcyclohexyl)malonate (5.9 g, 20.29 mmol, 68.1% yield) is obtained as a colorless liquid after distillation (P=0.3 mbar, T=75° C.).

Step 2: To a 250 mL three-necked flask under nitrogen are added dimethyl 2-((1R,2S,5R)-2-isopropyl-5-methylcyclohexyl)malonate (5 g, 18.49 mmol) et MTBE (71 ml). Sodium bis(2-methoxyethoxy)aluminum hydride 70% in toluene (26.4 ml, 92 mmol) is added dropwise while keeping the temperature below 10° C. The mixture is stirred at ambient temperature overnight. The mixture is carefully poured into a mixture of 10% aqueous HCl (100 mL) and ice. The aqueous phase is extracted with MTBE (2×50 mL) and the combined organic phases are washed with saturated aqueous NaHCO₃ (50 mL), brine (100 mL), dried over anhydrous MgSO₄, filtered and concentrated. 2-((1R,2S,5R)-2-isopropyl-5-methylcyclohexyl)propane-1,3-diol (1.8 g, 7.98 mmol, 43.1% yield) is recovered as a colorless liquid after recrystallization in refluxing cyclohexane (5 mL). ¹H NMR (300 MHz, CDCl₃) δ 3.94 (t, J=9.7 Hz, 1H), 3.87-3.75 (m, 1H), 3.75-3.62 (m, 2H), 3.02 (s, 2H), 2.21-2.10 (m, 1H), 2.10-1.94 (m, 1H), 1.74-1.59 (m, 2H), 1.57-1.44 (m, 1H), 1.39-1.30 (m, 1H), 1.30-1.18 (m, 1H), 1.13-1.01 (m, 1H), 1.00-0.94 (m, 1H), 0.91 (d, J=6.8 Hz, 3H), 0.84 (d, J=6.5 Hz, 4H), 0.75 (d, J=6.9 Hz, 3H), 0.68-0.53 (m, 1H). ¹³C NMR; IR (cm⁻¹); and Mass Spectrometry data were consistent with the expected data for the obtained compound. Olfactive description: Almost odorless, rosy.

### Compound 38: ((2S,5R)-1-((benzyloxy)methyl)-2-isopropyl-5-methylcyclohexyl) methanol

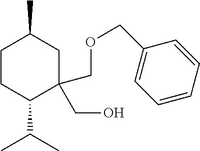 [SMILES: CC(C)[C@@H]1CC[C@@H](C)CC1(CO)COCc1ccccc1]

To a stirred solution of [(2S,5R)-1-(hydroxymethyl)-2-isopropyl-5-methyl-cyclohexyl]methanol (5.0 g, 25.0 mmol) in dry DMF (50 mL, [C]=0.5M) was added sodium hydride (60% in mineral oil, 1.05 g, 26.2 mmol, 1.05 eq.) portion wisely at 0° C. and the resulting mixture was stirred at 0° C. for 1h. Benzyl bromide (2.90 mL, 24.5 mmol, 0.98 eq.) was added dropwisely at 0° C. and the resulting mixture was stirred at room temperature for 20 h. Most of the DMF was removed under reduced pressure and the residue was partitioned between aq. Sat. NH₄Cl and EtOAc. The layers were separated and the aqueous phase was extracted with EtOAc. The combined organic extracts were washed with brine, dried over anhydrous Na₂SO₄, filtered and concentrated under reduced pressure. The residue (7.50 g) was purified by flash chromatography on silica gel (CyHex./EtOAc, 95/5) to afford [(2S,5R)-1-(benzyloxymethyl)-2-isopropyl-5-methyl-cyclohexyl]methanol (3.90 g, 51% yield) as a colourless oil. ¹H NMR (400 MHz, CDCl₃) δ 7.31-7.36 (m, 5H), 4.43-4.56 (m, 2H), 3.50-4.0 (m, 3H), 3.25-3.36 (m, 1H), 2.14-2.43 (m, 2H), 1.91-1.98 (m, 1H), 1.70-1.78 (m, 1H), 1.45-1.50 (m, 2H), 1.10-1.39 (m, 2H), 0.85-0.89 (m, 7H), 0.67-0.73 (m, 3H), 0.60-0.67 (m, 1H). Mass Spectrometry data was consistent with the expected data for the obtained compound. Olfactive description: Almost odorless, solvent.

### Compound 39: (2S,5R)-1-(hydroxymethyl)-2-isopropyl-5-methylcyclohexane-1-carboxylic acid

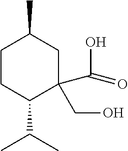

Step 1: To a stirred solution of [(2S,5R)-1-(benzyloxymethyl)-2-isopropyl-5-methyl-cyclohexyl]methanol (3.18 g, 10.9 mmol) in acetone (36 mL, [C]=0.3M) was added dropwisely Jones reagent (2.1M in water, 11.5 mL, 24.1 mmol, 2.2 eq.) at room temperature and the resulting mixture was stirred for 1h. The mixture was quenched by slow addition of iPrOH (5 mL), stirred for 30 min and concentrated under reduced pressure. The residue was taken up in water and extracted with Et₂O (3×30 mL). The combined organic extracts were dried over anhydrous Na₂SO₄, filtered and concentrated to afford (2S,5R)-1-(benzyloxymethyl)-2-isopropyl-5-methyl-cyclohexanecarboxylic acid (3.32 g, 90% yield) as a colourless oil. The crude mixture was used in the next step without further purification. HPLC/MS (acid): m/z (ES+)=327 (M+Na)⁺ Step 2: To a stirred solution of (2S,5R)-1-(benzyloxymethyl)-2-isopropyl-5-methyl-cyclohexanecarboxylic acid (1.50 g, 4.93 mmol) in dry DMF (16 mL, 0.3M) was added potassium carbonate (1.36 g, 9.85 mmol, 2 eq.) followed by benzyl bromide (0.88 mL, 7.39 mmol, 1.5 eq.) dropwisely at room temperature and the resulting mixture was stirred at RT for 20 h. Most of the DMF was removed under reduced pressure and the residue was poured into water. The aqueous layer was extracted with Et₂O (3×15 mL) and the combined organic extracts were washed with brine, dried over anhydrous Na₂SO₄, and concentrated. The crude mixture (2.15 g) was purified by flash chromatography on silica gel (CyHex./EtOAc, 99/1) to give benzyl (2S,5R)-1-(benzyloxymethyl)-2-isopropyl-5-methyl-cyclohexanecarboxylate, 1.73 g (80% yield) as a colorless oil. HPLC/MS (acid): m/z (ES+)=417 (M+Na)⁺.

Step 3: To a stirred solution of benzyl (2S,5R)-1-(benzyloxymethyl)-2-isopropyl-5-methyl-cyclohexanecarboxylate (1.73 g, 4.38 mmol) in iPrOH (22 mL, 0.2M) was added 10% palladium on carbon (93 mg, 0.877 mmol, 0.2 eq.) at room temperature under Ar. The mixture was flushed with hydrogen and stirred under hydrogen atmosphere at room temperature for 20 h. The mixture was filtered on a pad of Celite and the filtrate was concentrated under reduced pressure. The residue (1.08 g) was purified by flash chromatography on silica gel (CyHex./EtOAc, 9/1 to 1/1) to give (2S,5R)-1-(hydroxymethyl)-2-isopropyl-5-methyl-cyclohexanecarboxylic acid (550 mg, 56% yield) as a colorless oil which solidifies upon standing. ¹H NMR (400 MHz, CDCl₃) δ 4.14-4.17 (d, J=11.3 Hz, 1H), 4.05-4.08 (d, J=12 Hz, 1H), 3.68-3.71 (d, J=12 Hz, 1H), 3.32-3.35 (d, J=11.2 Hz, 1H), 2.14-2.43 (m, 2H), 1.67-1.86 (m, 4H), 1.55-1.57 (m, 2H), 0.92-0.94 (m, 1H), 0.75-0.92 (m, 9H). Mass Spectrometry data was consistent with the expected data for the obtained compound. Olfactive description: Almost odorless.

### Compound 40: (3,3,5,5-tetramethylcyclohexane-1,1-diyl)dimethanol

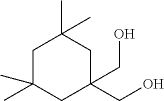

In a 50 ml three-necked flask are added potassium hydroxide (0.533 g, 9.51 mmol) and Ethanol (10 ml). After almost complete solubilization of the KOH, 37% aqueous formaldehyde (0.71 ml, 9.51 mmol) is added dropwise at ambient temperature, and then the mixture is stirred for 30 min. 23,3,5,5-tetramethylcyclohexane-1-carbaldehyde (0.4 g, 2.377 mmol) (synthetized according to WO2010025142) is then added dropwise over 1 min and the mixture is stirred 4 days at ambient temperature. The medium is concentrated and the aqueous phase extracted with MTBE (2×30 mL). The combined organic phases are washed sequentially with a saturated NaHCO₃ (10 mL) solution, and then brine (10 mL), dried over anhydrous MgSO₄, filtered, and concentrated. (3,3,5,5-tetramethylcyclohexane-1,1-diyl)dimethanol (0.3 g, 1.498 mmol, 63.0% yield) is recovered in the form of a white solid after purification by column chromatography (CombiFlash®, cyclohexane/EtOAc gradient). ¹H NMR (300 MHz, Chloroform-d) δ 3.59 (s, 4H), 3.43 (s, 2H), 1.22 (s, 2H), 1.19 (s, 4H), 0.99 (s, 12H). ¹³C NMR; IR (cm⁻¹); and Mass Spectrometry data were consistent with the expected data for the obtained compound. Olfactive description: Almost odorless, fresh.

### Evaluation of the Compounds In Vitro:

The compounds were evaluated in vitro on CHO-KJ cells expressing the gene encoding the hTRPM8 ion channel (Table 1). Channel activation is determined by fluorometric measurement of extracellular calcium using FLUO-8. The activity of the compounds was measured relative to icilin (known hTRPM8 agonist).

TABLE 1
Primary in vitro screening of various compounds on hTRPM8.

| | Concentration | % activation |
|---|---|---|
| Compound | (μM) | vs iciline |
| Compound 1 | 156 | 199 |
| Compound 2 | 109 | 26 |
| Compound 3 | 104 | 99 |
| Compound 4 | 101 | 39 |
| Compound 5 | 96 | 176 |
| Compound 6 | 104 | 32 |
| Comparison 1: Physcool ™ | 105 | -2 |
| Comparison 2: WS-5 ™ | 105 | 73 |
| Comparison 3: WS-3 ™ | 119 | 76 |
| Comparison 4: l-isopulegol | 123 | 51 |
| Comparison 5: l-menthol | 114 | 78 |
| Comparison 6: menthyl lactate | 116 | 53 |

Compounds 1 to 6 were tested and shown to be active on hTRPM8, and certain inventive compounds (1, 3 and 5) show greater activations than the comparative molecules (Physcool™, WS-5™, WS-3™, l-isopulegol, l-menthol, menthyl lactate).

The compounds 1 to 40 were tested on hTRPM8 at 5 μM using WS3 as the reference compound (Table 2).

TABLE 2
Secondary in vitro screening of the compounds
on hTRPM8 at 5 μM versus WS-3.

| Compound | Activity* |
|---|---|
| 1 | 99 |
| 2 | 3 |
| 3 | 94 |
| 4 | −1 |
| 5 | 16 |
| 6 | 3 |
| 7 | 0 |
| 8 | 6 |
| 9 | 0 |
| 10 | 10 |
| 11 | 3 |
| 12 | 1 |
| 13 | 1 |
| 14 | −1 |
| 15 | −6 |
| 16 | −1 |
| 17 | 1 |
| 18 | 3 |
| 19 | 10 |
| 20 | 4 |
| 21 | −2 |
| 22 | 13 |
| 23 | 4 |
| 24 | 3 |
| 25 | 3 |
| 26 | 3 |
| 27 | 40 |
| 28 | 5 |
| 29 | 26 |
| 30 | 71 |
| 31 | −2 |
| 32 | 4 |
| 33 | 1 |
| 34 | 21 |
| 35 | 14 |
| 36 | 6 |
| 37 | 65 |
| 38 | 8 |
| 39 | 16 |
| 40 | 8 |

*Activity @5 μM vs WS3 (%)

A response curve was developed for compounds 1, 3, 5 and 37 which made it possible to determine the EC50 (half maximal effective concentration) values on hTRPM8 and compare them with the EC50 values of the comparative compounds (Table 3). Compound 1 and compound 3 have EC50 values lower than WS-3™ and similar to WS-5™

TABLE 3
EC50 measurement on hTRPM8.

| Molecule | EC50 (μM) |
|---|---|
| Compound 1 | 4.6 |
| Compound 3 | 2.5 |
| Compound 5 | 39 |
| Compound 37 | 70 |
| Comparison 1: WS-5 ™ | 1 |
| Comparison 2: WS-3 ™ | 8 |

### Evaluation of Compounds In Vivo:

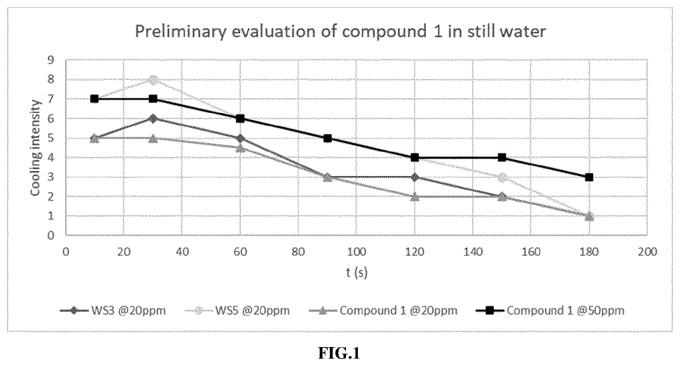

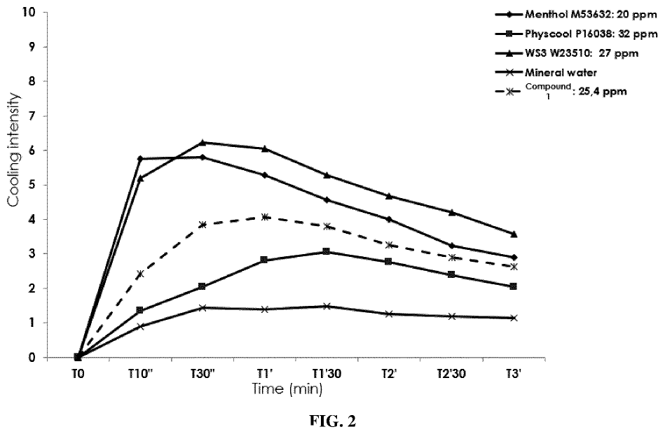

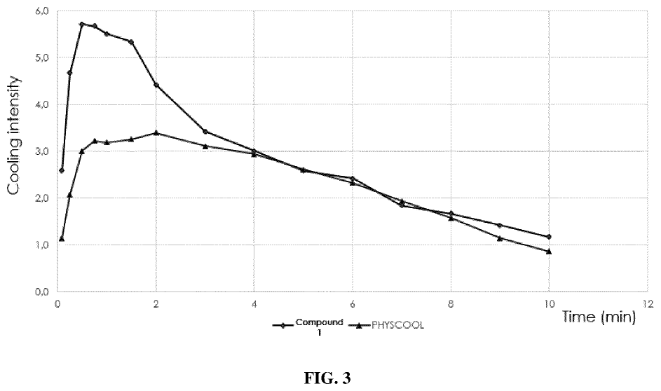

Inventive compounds 1, 2 and 5 were organoleptically evaluated in still water by a trained panel. The compound 1 particularly demonstrated freshness in the mouth with both an attack and a good persistence. An evaluation against comparative compounds WS-3™ and WS-5™ was made (FIG. 1). These inventive compounds appear to behave similarly to the comparative compounds. Equimolar comparative analysis of some inventive compounds showed better performance than Physcool™ (FIG. 2). An equimolar comparative analysis on a larger panel and over 10 min shows that the Compound 1 has a persistent cooling effect similar to Physcool® but with a greater intensity (FIG. 3).

### EXAMPLE 1: Chewing Gum

An unflavored chewing gum paste was prepared using the following formulation: Sorbitol (52.4 wt %), gum base (30.0 wt %), maltitol syrup (7.0 wt %), glycerine (5.0 wt %), mannitol (5.0 wt %), soy lecithin (0.4 wt %), acesulfame-K (0.1 wt %), and aspartame (0.1 wt %). The paste was warmed 1-2 min using a micro-wave to soften the mixture. After adding the cooling agent (0.20 wt % Compound 1), the paste was kneaded to homogenise the product. Then the paste is stretched using a sheeter, flatted at 2 mm thick, and cut in regular rectangular pieces (12×42 mm). Chewing-gums pieces are packed in plastic bag and put 1 week at room temperature to let them stabilise. Comparative chewing gum samples (A-C) were also prepared using 0.26 wt % PHYSCOOL™; 0.21 wt % WS-3™; and 0.17 wt % WS-23™, respectively.

A chewing gum evaluation protocol was performed by 6 trained panelists, where the chewing gum was chewed for 10 min while holding the panelist's mouth closed. The cooling intensity was rated from 0 (not perceived) to 10 (very intense) at 5 sec, 15 sec, 30 sec, 45 sec, 1 min, 1.5 min, 2 min, and then every minute until 10 minutes. On average, the panelist found the chewing gum comprising inventive Compound 1 to have more initial cooling intensity and longer lasting duration than Comparative Sample A (PHYSCOOL™). The panelist also found that the inventive Compound 1 provide long cooling intensity out to 10 minutes, similar to that provided by WS-3™ in Comparative Example B and WS-23™ in Comparative Example C.

While the invention has been illustrated by the description of one or more embodiments thereof, and while the embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative product and/or method and examples shown and described. The various features of exemplary embodiments described herein may be used in any combination. Accordingly, departures may be made from such details without departing from the scope of the general inventive concept.

1. A cooling composition comprising:
an effective amount of a cooling compound having a general Formula (I): 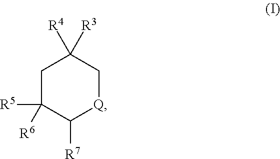
wherein Q is selected from CR¹R² or CH-CH(CH₂OR⁸)CH₂OR⁹; wherein R¹ and R² are independently selected from the group consisting of CHO, CO₂H; CH₂CH₂OH; CHR¹⁰R¹¹, and CHR¹⁰R¹²; wherein R⁸ and R⁹ are independently H, or in combination form a spiroacetal or spiroketal moiety; wherein each R¹⁰ is independently H or CH₃; wherein R¹¹ and R¹² are independently selected from H, substituted or unsubstituted C1 to C6 alkyls, substituted or unsubstituted cycloalkyls, substituted or unsubstituted alkaryls, substituted or unsubstituted aryls or heteroaryls, and substituted or unsubstituted acyls, or wherein R¹¹ and R¹² in combination form a spiroketal or a spiroacetal moiety;
wherein R³, R⁴, R⁵, and R⁶ are independently selected from H, CH₃, and CH₂CH₃; and wherein R⁷ is selected from H, iso-propyl, isopropenyl, and sec-butyl; with the proviso that R³, R⁴, R⁵, R⁶, and R⁷ are not all H; and
an edible, potable, or cosmetic ingredient.

2. The cooling composition as claimed in claim 1, wherein Q is CR¹ R² in Formula (I), thereby the cooling compound having a general Formula (II): 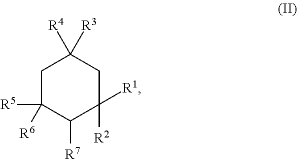
wherein R¹-R⁷ are the same as defined with respect to Formula (I) in claim 1.

3. The cooling composition as claimed in claim 2, wherein R³, R⁵, and R⁶ are each H; wherein R⁴ is methyl; and wherein R⁷ is isopropyl in Formula (II), thereby the cooling compound having a general Formula (III), Formula (IVₐ), or Formula (IV_{b}):

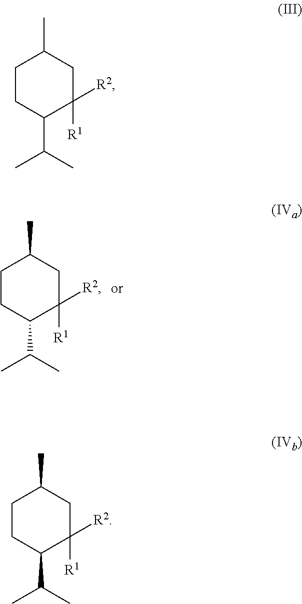

4. The cooling composition as claimed in claim 1, wherein R¹ and R² are each CHO.

5. The cooling composition as claimed in claim 2, wherein R is CHR¹⁰OR¹¹, R² is CHR¹⁰OR¹² in Formula (II); and wherein R¹ is H, thereby the cooling compound having a general Formula (VIII):

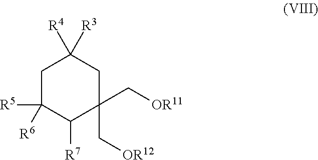

6. The cooling composition as claimed in claim 5, wherein R¹¹ and R¹² in Formula (VIII) in combination form a spiroketal or a spiroacetal moiety CR¹³R¹⁴, thereby the cooling compound having a general Formula (IX): 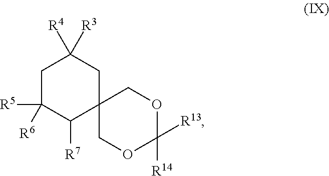
wherein R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls, or R¹³ and R¹⁴ in combination form a substituted or unsubstituted cycloalkyl.

7. The cooling composition as claimed in claim 3, wherein R¹ is CHR¹⁰R¹¹, and R² is CHR¹⁰R¹² in Formulas (III), (IVa), and (IVb), thereby the cooling compound having a general 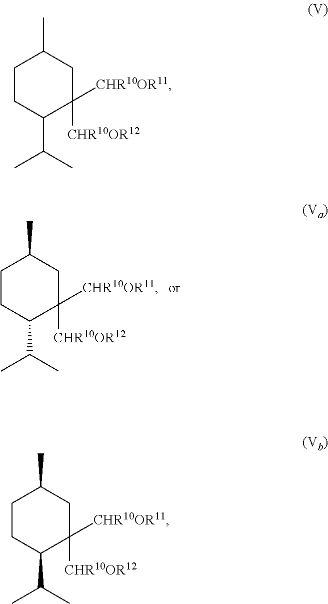
wherein R¹¹ and R¹² are independently selected from H, substituted or unsubstituted C1 to C6 alkyls; substituted or unsubstituted cycloalkyls; substituted or unsubstituted alkaryls; substituted or unsubstituted aryls or heteroaryls; and substituted or unsubstituted acyls; or
wherein R¹⁰ is H, and R¹¹ and R¹² in combination form a spiroketal or a spiroacetal moiety CR¹³R¹⁴, thereby the cooling compound having a general Formula (VI), (VIₐ), or (VI_{b}): 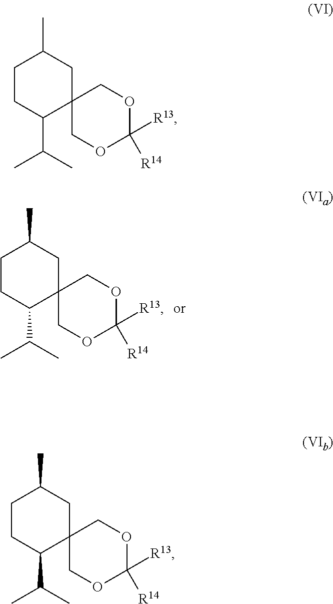
wherein R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls; or wherein R¹³ and R¹⁴ in combination form a substituted or unsubstituted cycloalkyl.

8. The cooling composition as claimed in claim 2, wherein R³, R⁴, and R⁵ are each methyl, and wherein R⁶ and R⁷ are both H in Formula (II), thereby the cooling compound having a general Formula (X):

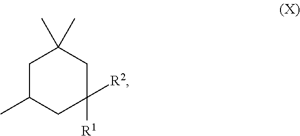

9. The cooling composition as claimed in claim 8, wherein R is CHR¹⁰R¹¹, R² is CHR¹⁰R¹² in Formula (X), and wherein R¹⁰ is H, thereby the cooling compound having a general Formula (XI): 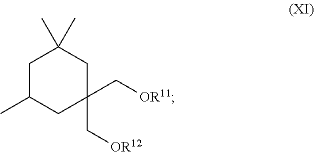
wherein R¹¹ and R¹² optionally form a spiroketal or a spiroacetal moiety CR¹³R¹⁴, thereby the cooling compound having a general Formula (XII): 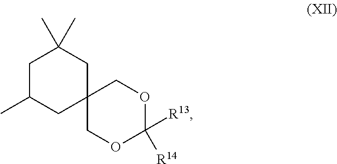
where R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls; or wherein R¹³ and R¹⁴ in combination form a substituted or unsubstituted cycloalkyl.

10. The cooling composition as claimed in claim 1, where Q is CH-CH(CH₂OR⁸) CH₂OR⁹ in Formula (I), thereby the cooling compound having a general Formula (VII):

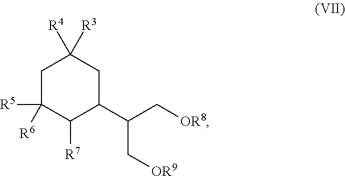

11. The cooling composition as claimed in claim 1, wherein the cooling compound is selected from the group consisting of: ((2S, 5R)-2-isopropyl-5-methylcyclohexane-1, 1-diyl)dimethanol; (7S, 1OR)-7-isopropyl-3, 10-dimethyl-2,4-dioxaspiro[5.5]undecane; (7S,1OR)-7-isopropyl-3,3,10-trimethyl-2,4-dioxaspiro[5.5]undecane; (7S,1OR)-3-ethyl-7-isopropyl-10-methyl-2,4-dioxaspiro[5.5]undecane; 4-((7S,10R)-7-isopropyl-10-methyl-2,4-dioxaspiro[5.5]undecan-3-yl)-2-methoxyphenol; (7S, 1OR)-7-isopropyl-10-methyl-2,4-dioxaspiro[5.5]undecane; (1 S,4R, 1 OS, 13R)-1, 10-diisopropyl-4,13-dimethyl-7,16-dioxadispiro[5.2.59.26]hexadecane; (2S,5R)-2-isopropyl-5-methylcyclohexane-1, 1-dicarbaldehyde; ((2S,5R)-2-isopropyl-5-methylcyclohexane-1,1-diyl)bis(methylene) diacetate; (3,3,5-trimethylcyclohexane-1,1-diyl)dimethanol; 3,8,8,10-tetramethyl-2,4-dioxaspiro[5.5]undecane; 3,3,8,8,10-pentamethyl-2,4-dioxaspiro[5.5]undecane; (7S,1OR)-3-ethyl-7-isopropyl-3,10-dimethyl-2,4-dioxaspiro[5.5]undecane; (7S,1OR)-7-isopropyl-10-methyl-3-propyl-2,4-dioxaspiro[5.5]undecane; (7S,1OR)-3,7-diisopropyl-10-methyl-2,4-dioxaspiro[5.5]undecane; (7S,1OR)-3-(sec-butyl)-7-isopropyl-10-methyl-2,4-dioxaspiro[5.5]undecane; (2S,5R)-2-isopropyl-1,1-bis(methoxymethyl)-5-methylcyclohexane; (7S,1OR)-7-isopropyl-10-methyl-3-phenyl-2,4-dioxaspiro[5.5]undecane; (7S,1OR)-3,3-diethyl-7-isopropyl-10-methyl-2,4-dioxaspiro[5.5]undecane; (3,3-dimethylcyclohexane-1,1-diyl)dimethanol; 4,4′-((((2S,5R)-2-isopropyl-5-methylcyclohexane-1,1-diyl)bis(methylene))bis(oxy))bis(4-oxobutanoic acid)(7S, 1OR)-7-isopropyl-3, 10-dimethyl-3-phenyl-2,4-dioxaspiro[5.5]undecane; 2-((7S, 1OR)-7-isopropyl-10-methyl-2,4-dioxaspiro[5.5]undecan-3-yl)pyridine; (7S,1OR)-7-isopropyl-10-methyl-3-(thiophen-2-yl)-2,4-dioxaspiro[5.5]undecane; (7S,1OR)-7-isopropyl-10-methyl-3-(2-(methylthio)ethyl)-2,4-dioxaspiro[5.5]undecane; 3,8,8-trimethyl-2,4-dioxaspiro[5.5]undecane; (2-(sec-butyl)cyclohexane-1, 1-diyl)dimethanol; ethyl (7S, 1OR)-7-isopropyl-10-methyl-2,4-dioxaspiro[5.5]undecane-3-carboxylate; 2,2′-((2S,5R)-2-isopropyl-5-methylcyclohexane-1,1-diyl)bis(ethan-1-ol); (2-isopropyl-5,5-dimethylcyclohexane-1,1-diyl)dimethanol; 7-(sec-butyl)-3-methyl-2,4-dioxaspiro[5.5]undecane; ((2S,5R)-1-(hydroxymethyl)-2-isopropyl-5-methylcyclohexyl)methyl acetate; 7-isopropyl-3, 10, 10-trimethyl-2,4-dioxaspiro[5.5]undecane; 1, 1′-((2S,5R)-2-isopropyl-5-methylcyclohexane-1, 1-diyl)bis(ethan-1-ol); 2-((1R,2R,5R)-5-methyl-2-(prop-1-en-2-yl)cyclohexyl)propane-1,3-diol; 2-methyl-5-((1R,2R,5R)-5-methyl-2-(prop-1-en-2-yl)cyclohexyl)-1,3-dioxane; 2-((1R,2S,5R)-2-isopropyl-5-methylcyclohexyl)propane-1,3-diol; ((2S,5R)-1-((benzyloxy)methyl)-2-isopropyl-5-methylcyclohexyl)methanol; (2S,5R)-1-(hydroxymethyl)-2-isopropyl-5-methylcyclohexane-1-carboxylic acid; (3,3,5,5-tetramethylcyclohexane-1,1-diyl)dimethanol; and mixtures thereof.

12. The cooling composition as claimed in claim 1, further comprising: a secondary coolant agent selected from the group consisting of carboxamides, ketals, menthyl glutarate, menthyl succinate, menthyl acetate, menthyl lactate, 3-menthoxypropane-1,2 diol, isopulegol, menthol, and mixtures thereof.

13. A product composition selected from topical, ingestible, or tobacco products, such as oral care products, nasal care products, toiletries, filters, combustible papers and coating sheets for smoking tobacco products, chewing tobacco, chewing tobacco products, snuff tobacco products, chewing gum and chewing gum products, comprising:
a topical, ingestible, or tobacco base product, and
an effective amount of the cooling composition as claimed claim 1.

14. A cooling compound having a general Formula (I): 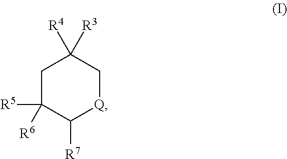
wherein Q is selected from CR¹R² or CH-CH(CH₂OR⁸)CH₂OR⁹; wherein R¹ is selected from the group consisting of CHO, CO₂H; CH₂CH₂OH; CHR¹⁰R¹¹; wherein R² is selected from the group consisting of CHO, CH₂CH₂OH, and CHR¹⁰R¹²; wherein R¹ and R⁹ are independently H, or in combination form a spiroacetal or spiroketal moiety; wherein R¹⁰ is H or CH₃; wherein R¹¹ and R¹² are independently selected from H, substituted or unsubstituted C1 to C6 alkyls, substituted or unsubstituted cycloalkyls, substituted or unsubstituted alkaryls, substituted or unsubstituted aryls or heteroaryls, and substituted or unsubstituted acyls, or wherein R¹¹ and R¹² in combination form a spiroketal or a spiroacetal moiety; and
wherein R³, R⁴, R⁵, and R⁶ are independently selected from H, CH₃, and CH₂CH₃; where R⁷ is selected from iso-propyl, isopropenyl, and sec-butyl;
with a first proviso that R³, R⁴, R⁵, R⁶, and R⁷ are not all H;
with a second proviso that when Q is CR¹R², and R¹ and R² are both CH₂OH:
then R⁷ is not H; and
then R³, R⁵, and R⁶ are not all H, R⁴ is not CH₃, and R⁷ is not isopropyl;
with a third proviso that when Q is CR¹R², R¹ and R² are both CH₂OH, and R⁷ is isopropyl or isobutyl, then at least two or more of R³, R⁴, R⁵, and R⁶ are CH₃ or CH₂CH₃; and
with a fourth proviso that when Q is CR¹R², and R¹ is CHR¹⁰R¹¹, R² is CHR¹⁰R¹², R¹⁰ is H, and R¹¹ and R¹² in combination form a spiroketal or a spiroacetal moiety; and R⁷ is H, then at least two or more of R³, R⁴, R⁵, and R⁶ are CH₃ or CH₂CH₃.

15. The cooling compound as claimed in claim 14, where Q is CR¹R² in Formula (I), thereby the cooling compound having a general Formula (II): 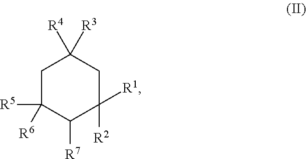
wherein R¹-R⁷ are the same as defined with respect to Formula (I) in claim 14.

16. The cooling compound as claimed in claim 15, wherein R³, R⁵, and R⁶ are each H, R⁴ is methyl, and R⁷ is isopropyl in Formula (II), thereby the cooling compound having a general formula:

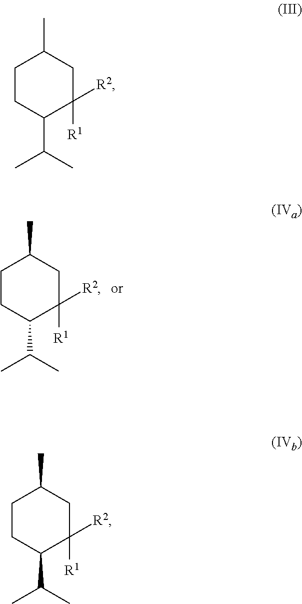

17. The cooling compound as claimed in claim 14, wherein R¹ and R² are each CHO.

18. The cooling compound as claimed in claim 15, wherein R¹ is CHR¹⁰R¹¹, and R² is CHR¹⁰OR¹² in Formula (II), and R¹⁰ is H, thereby the cooling compound having a general Formula (VIII):

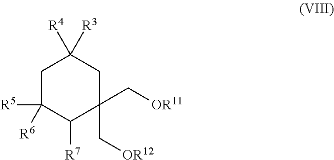

19. The cooling compound as claimed in claim 18, wherein R¹¹ and R¹² in combination form a spiroketal or a spiroacetal moiety CR¹³R¹⁴, thereby the cooling compound having a general Formula (IX): 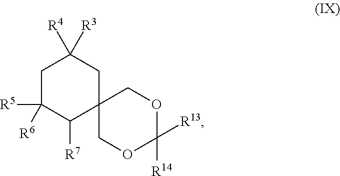
wherein R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls; or R¹³ and R¹⁴ in combination form a substituted or unsubstituted cycloalkyl.

20. The cooling compound as claimed in claim 16, wherein R¹ is CHR¹⁰R¹¹, and R² is CHR¹⁰OR¹² in Formulas (III), (IVₐ), and (IV_{b}), thereby the cooling compound having a general 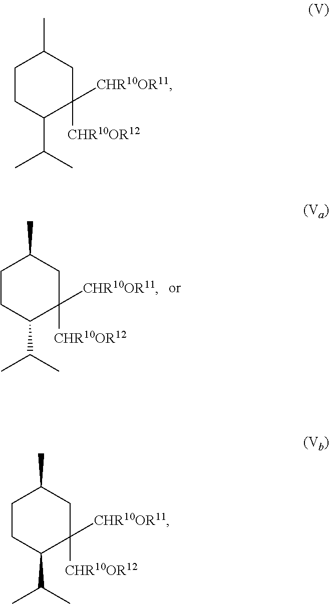
where R¹¹ and R¹² are independently selected from H, substituted or unsubstituted C1 to C6 alkyls; substituted or unsubstituted cycloalkyls; substituted or unsubstituted alkaryls; substituted or unsubstituted aryls or heteroaryls; and substituted or unsubstituted acyls; or
wherein R¹⁰ is H, and R¹¹ and R¹² in combination form a spiroketal or a spiroacetal moiety CR¹³R¹⁴, thereby the cooling compound having a general Formula (VI), (VIₐ), or (VI_{b}): 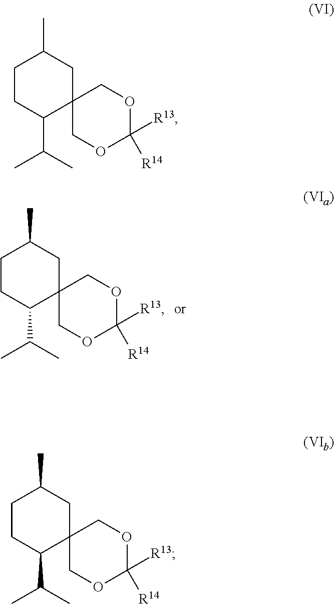
wherein R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls; or R¹³ and R¹⁴ in combination form a substituted or unsubstituted cycloalkyl.

21. The cooling compound as claimed in claim 15, wherein R³, R⁴, and R⁵ are each methyl, wherein R⁶ and R⁷ are both H in Formula (II), thereby the cooling compound having a general Formula (X):

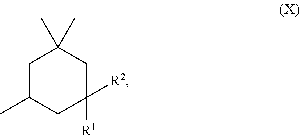

22. The cooling compound as claimed in claim 21, wherein R′ is CHR¹⁰R¹¹, R² is CHR¹⁰OR¹² in Formula (X), and wherein R¹⁰ is H, thereby providing a cooling compound having a general Formula (XI); 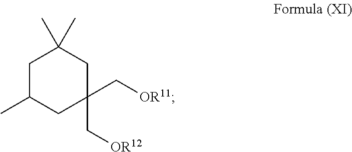
wherein R¹¹ and R¹² are optionally in combination form a spiroketal or a spiroacetal moiety CR¹³R¹⁴, thereby the cooling compound having a general Formula (XII): 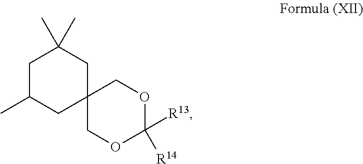
where R¹³ and R¹⁴ are independently selected from the group consisting of H, substituted or unsubstituted C1 to C6 alkyls; substituted or unsubstituted cycloalkyls; substituted or unsubstituted alkaryls; and substituted or unsubstituted aryls or heteroaryls; or R¹³ and R¹⁴ in combination form a substituted or unsubstituted cycloalkyl.

23. The cooling compound as claimed in claim 14, where Q is CH-CH(CH₂OR⁸) CH₂OR⁹ in Formula (I), thereby the cooling compound having a general Formula (VII): 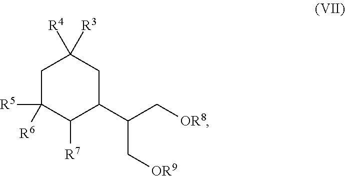
wherein R³ to R⁹ are the same as defined with respect to Formula (I) in claim 14.

24. The cooling compound as claimed in claim 14, wherein the cooling compound is selected from the group consisting of: ((2S, 5R)-2-isopropyl-5-methylcyclohexane-1, 1-diyl)dimethanol; (7S, 10R)-7-isopropyl-3, 10-dimethyl-2,4-dioxaspiro[5.5]undecane; (7S, 1OR)-7-isopropyl-3,3, 10-trimethyl-2,4-dioxaspiro [5.5]undecane; (7S, 1OR)-3-ethyl-7-isopropyl-10-methyl-2,4-dioxaspiro[5.5]undecane; 4-((7S,10R)-7-isopropyl-10-methyl-2,4-dioxaspiro[5.5]undecan-3-yl)-2-methoxyphenol; (7S,1OR)-7-isopropyl-10-methyl-2,4-dioxaspiro[5.5]undecane; (1S,4R,10S,13R)-1,10-diisopropyl-4,13-dimethyl-7, 16-dioxadispiro[5.2.59.26]hexadecane; (2S,5R)-2-isopropyl-5-methylcyclohexane-1, 1-dicarbaldehyde; ((2S,5R)-2-isopropyl-5-methylcyclohexane-1, 1-diyl)bis(methylene) diacetate; 3,8,8, 10-tetramethyl-2,4-dioxaspiro[5.5]undecane; 3,3,8,8,10-pentamethyl-2,4-dioxaspiro[5.5]undecane; (7S,1OR)-3-ethyl-7-isopropyl-3,10-dimethyl-2,4-dioxaspiro [5.5]undecane; (7S,1OR)-7-isopropyl-10-methyl-3-propyl-2,4-dioxaspiro[5.5]undecane; (7S,1OR)-3,7-diisopropyl-10-methyl-2,4-dioxaspiro[5.5]undecane; (7S,1OR)-3-(sec-butyl)-7-isopropyl-10-methyl-2,4-dioxaspiro[5.5]undecane; (2S,5R)-2-isopropyl-1,1-bis(methoxymethyl)-5-methylcyclohexane; (7S,1OR)-7-isopropyl-10-methyl-3-phenyl-2,4-dioxaspiro[5.5]undecane; (7S,1OR)-3,3-diethyl-7-isopropyl-10-methyl-2,4-dioxaspiro[5.5]undecane; 4,4′-((((2S,5R)-2-isopropyl-5-methylcyclohexane-1,1-diyl)bis(methylene))bis(oxy))bis(4-oxobutanoic acid); (7S, 1OR)-7-isopropyl-3, 10-dimethyl-3-phenyl-2,4-dioxaspiro[5.5]undecane; 2-((7S,10R)-7-isopropyl-10-methyl-2,4-dioxaspiro[5.5]undecan-3-yl)pyridine; (7S,1OR)-7-isopropyl-10-methyl-3-(thiophen-2-yl)-2,4-dioxaspiro[5.5]undecane; (7S, 1OR)-7-isopropyl-10-methyl-3-(2-(methylthio)ethyl)-2,4-dioxaspiro[5.5]undecane; 3,8,8-trimethyl-2,4-dioxaspiro[5.5]undecane; ethyl (7S,1OR)-7-isopropyl-10-methyl-2,4-dioxaspiro[5.5]undecane-3-carboxylate; 2,2′-((2S,5R)-2-isopropyl-5-methylcyclohexane-1, 1-diyl)bis(ethan-1-ol); (2-isopropyl-5,5-dimethylcyclohexane-1,1-diyl)dimethanol; 7-(sec-butyl)-3-methyl-2,4-dioxaspiro [5.5]undecane; ((2S,5R)-1-(hydroxymethyl)-2-isopropyl-5-methylcyclohexyl)methyl acetate; 7-isopropyl-3, 10, 10-trimethyl-2,4-dioxaspiro[5.5]undecane; 1, 1′-((2S,5R)-2-isopropyl-5-methylcyclohexane-1, 1-diyl)bis(ethan-1-ol); 2-((1R,2R,5R)-5-methyl-2-(prop-1-en-2-yl)cyclohexyl)propane-1,3-diol; 2-methyl-5-((1R,2R,5R)-5-methyl-2-(prop-1-en-2-yl)cyclohexyl)-1,3-dioxane; 2-((1R,2S,5R)-2-isopropyl-5-methylcyclohexyl)propane-1,3-diol; ((2S,5R)-1-((benzyloxy)methyl)-2-isopropyl-5-methylcyclohexyl)methanol; (2S,5R)-1-(hydroxymethyl)-2-isopropyl-5-methylcyclohexane-1-carboxylic acid; and mixtures thereof.

25. Use of the cooling compound as defined in claim 1 as a cooling agent or a perfume agent.
